(19) United States
(12) Patent Application Publication
(10) Pub. No.: US 2025/0230312 A1
(43) Pub. Date: Jul. 17, 2025

(54) MULTI-TRANSITION SHAPE MEMORY POLYMER AND METHODS OF PREPARATION

(71) Applicant: The Curators of the University of Missouri, Columbia, MO (US)

(72) Inventors: JIAN LIN, Columbia, MO (US); ALIREZA MAHJOUBNIA, Columbia, MO (US)

(21) Appl. No.: 19/027,205

(22) Filed: Jan. 17, 2025

Related U.S. Application Data
(60) Provisional application No. 63/621,784, filed on Jan. 17, 2024.

(51) Int. Cl. C08L 33/12

(52) U.S. Cl. CPC C08L 33/12; C08L 2201/06; C08L 2203/02

(57) ABSTRACT

Described herein are shape memory elastomers (SMEs) each comprising a copolymer network structure. The network comprises two distinct polymer components: a first polymer that is a biocompatible, biodegradable shape memory polymer exhibiting hydrophobic properties, and a second polymer that is covalently bonded to the first via the covalent or/and noncovalent interactions. This second polymer is distinguished by its biocompatible, biodegradable, and hydrophilic nature, featuring functional groups with hydrogen bonding capabilities. The network can also incorporate a third polymer, resulting in an SME with multiple shape transition properties. The SMEs described herein have unique potential as biomedical implants. Methods of manufacture are also provided.

### CROSS-REFERENCE TO RELATED APPLICATIONS

This application claims priority to U.S. Provisional Application No. 63/621,784 filed on Jan. 17, 2024, the content of which (text, drawings, and claims) is incorporated herein by reference.

### GOVERNMENT RIGHTS

This invention was made with government support under EY034254 awarded by the National Institutes of Health. The government has certain rights in the invention.

### TECHNICAL FIELD

The present teachings relate to four-dimensional printing, shape memory elastomers, minimally invasive surgery, and biomedical devices.

### BACKGROUND

Shape memory elastomers (SMEs) are materials that can undergo reversible transformations between desired pre-programmed shapes in response to external stimuli while maintaining significant elasticity. SMEs can be ‘programmed’ to transform between specific desired shapes, where the term ‘programming’ refers to a process of deforming the materials at elevated temperatures and fixing their shape by cooling. SMEs combine the programmable nature of shape memory polymers with the mechanical resilience of elastomers, offering unique possibilities for applications such as biomedical implantation that require transformation between particular shapes and mechanical robustness. However, developing SMEs that are appropriately mechanically robust and that transform only in response to very specific external stimuli has proven challenging. For use as biomedical implants, for example, SMEs must exhibit transition temperatures compatible with physiological temperatures while maintaining their physical integrity, chemical stability, and position in vivo. However, many known SMEs transform at unsuitable transition temperatures, show poor mechanical properties such as brittleness, and are difficult to make sufficiently resilient, positionally stable, and biocompatible.

The field of SMEs particularly struggles with maintaining mechanical integrity during shape recovery and usage, as many SMEs show dramatically reduced strength and toughness above their transition temperature. This compromise between shape memory functionality and mechanical performance has limited their practical applications. Additionally, achieving precise control over transition temperatures while maintaining elastomeric properties has remained elusive, with most materials exhibiting transitions either well below room temperature or far above physiological temperatures.

In the context of biomedical implants, these challenges become even more critical. Modern minimally invasive surgery demands materials that can be compacted for delivery yet expand to complex, predetermined shapes once in place. While various biomedical implants, from cardiovascular stents to reconstructive devices, could benefit from shape-memory capabilities, the materials must also exhibit tissue-like mechanical properties and maintain structural integrity under physiological conditions, such as the temperatures, pressures, and salinities present in various parts of the human body. Additional requirements such as biocompatibility and, in some cases, controlled degradation of biomedical implants that are intended to slowly disintegrate in the human body, further complicate the material design space.

Conventional SMEs often fall short in meeting these demanding criteria, particularly in their inability to maintain sufficient mechanical properties above their transition temperature—a crucial requirement for reliable deployment in the body. There is thus an urgent need for materials that can combine programmable shape memory behavior, robust mechanical properties across their operating temperature range, and biological compatibility, potentially including controlled degradation in vivo. Such materials would not only advance minimally invasive surgical techniques but could also enable new therapeutic approaches in tissue engineering and regenerative medicine.

### BRIEF SUMMARY

Described herein is a shape memory elastomer (SME). The SME comprises a copolymer network that is built from a first polymer, which is a biocompatible, biodegradable shape memory polymer that exhibits hydrophobic character. The first polymer is bonded to a second polymer, which is both biocompatible and biodegradable, is hydrophilic, and contains one or more functional groups that can form hydrogen bonds.

In a non-limiting sense, the first polymer can in various exemplary embodiments be selected from several options, including poly(glycerol dodecanoate acrylate) (PGDA), polyurethane, polycaprolactone, polylactic acid, polyethylene, polystyrene, polynorborene, polymethyl methacrylate, polyvinyl chloride, poly(ethylene-co-vinyl acetate), and polyimide. In a non-limiting sense the functional groups on the second polymer can, in various exemplary embodiments, be carboxylic acids, hydroxyls, amides, or phenols. In a non-limiting sense, in various exemplary embodiments, the second polymer can be chosen from polyacrylic acid (PAA), polyvinyl alcohol, polyethylene glycol, polyethylene glycol diacrylate, polyacrylamide, polymethacrylic acid, hyaluronic acid, chitosan, tannic acid, polyitaconic acid, maleic acid, alginate, peptide, chitosan, or collagen.

In various exemplary embodiments, the first polymer is poly(glycerol dodecanoate acrylate) (PGDA) and the second polymer is polyacrylic acid (PAA), forming a PGDA-PAA copolymer network. This network can be constructed with a PGDA:PAA weight ratio of 70:30. In various exemplary embodiments, the SME can undergo a shape change transition at a transition temperature Tₜᵣₐₙₛ that falls between 30° C. and 50° C., for example approximately 39° C.

The SME can further include a third polymer, which is biodegradable and biocompatible but different from the first polymer. In various exemplary embodiments, all three polymers are connected to each other. In a non-limiting sense, the third polymer can be selected from 10-undecenoic acid (UA), polyurethane, polycaprolactone, polylactic acid, polyethylene, polystyrene, polynorborene, polymethyl methacrylate, polyvinyl chloride, poly(ethylene-co-vinyl acetate), or polyimide. In various exemplary embodiments, the three polymers form a PGDA-PUA-PAA copolymer network with a weight ratio of 50:20:30.

In various exemplary embodiments an object can comprise the SME and exhibit unique shape-memory properties. The object has an original shape established during synthesis and can, in various exemplary embodiments, be deformed into multiple different programmed shapes depending on temperature conditions. For example, the object can assume a first programmed shape above a first transition temperature but below a second transition temperature, and can take on a second programmed shape below the first transition temperature. The object returns to its original shape or to a recovered shape, where the recovered shape is approximately the same as the original shape, when heated above the second transition temperature, which is higher than the first transition temperature.

In various exemplary embodiments the first transition temperature for the object is below 30° C. and the second transition temperature is above 35° C. When exposed to water, the object can absorb and retain it until the object becomes a hydrogel, which affects the object's transition temperatures, such that both first and second transition temperatures decrease as water absorption increases. In various exemplary embodiments, when the object is in a dry state, the second transition temperature remains above internal body temperature (for example above 40° C.), but drops below internal body temperature when the object is in hydrogel form. In various exemplary embodiments the hydrogel state causes significant volume expansion, with the object reaching at least ten times the dry volume.

Finally, the SME can be incorporated into a biomedical implant. The biomedical implant retains an original shape set during synthesis that, in various exemplary embodiments, is specifically designed to support bodily tissue at a treatment site in a patient. The implant is engineered to be deliverable via a catheter to the treatment site.

Methods of manufacturing the SME include molding, extrusion, machining, extrusion-based 3D printing, and light-based 3D printing, and any other method known to one of ordinary skill in the art.

### BRIEF DESCRIPTION OF THE FIGURES

FIG. 1A exemplarily depicts the PGDA-PAA network of the biocompatible SME in accordance with various embodiments of the present disclosure.

FIG. 1B exemplarily depicts a molecular-level view the PGDA-PAA network of the biocompatible SME in accordance with various embodiments of the present disclosure.

FIG. 2 exemplarily provides a schematic depiction of shape programming and restoration of the original shape of the biocompatible SME in accordance with various embodiments of the present disclosure.

FIG. 3A exemplarily depicts the PGDA-PUA-PAA network of the multi-transition SME in accordance with various embodiments of the present disclosure.

FIG. 3B exemplarily depicts a molecular-level view of the PGDA-PUA-PAA network of the multi-transition SME in accordance with various embodiments of the present disclosure.

FIG. 4 exemplarily provides a schematic depiction of shape programming and restoration of the original shape of the multi-transition SME in accordance with various embodiments of the present disclosure.

FIG. 5 exemplarily provides DSC curves of exemplary dry and hydrated PGDA-PUA-PAA networks (weight ratio: 50:20:30) of multi-transition SMEs in accordance with various embodiments of the present disclosure.

FIG. 6 exemplarily provides a schematic showing a full programming and deployment cycle of a delayed-onset implant from an original to a programmed shape at room temperature, and finally to a hydrogel at body temperature (>Tₜᵣₐₙₛ₂).

FIG. 7 exemplarily shows rheological changes in pre-PGDA resins of various weight percentages for consideration in synthesizing PGDA-PAA networks in accordance with various embodiments of the present disclosure.

FIGS. 8A, 8B, and 8C exemplarily show vibrational spectra indicating variations in the extent of crosslinking in pre-PGDA resin, inks of varying pre-PGDA weight content, and exemplary PGDA-PAA SME before and after UV exposure of varying times in accordance with various embodiments of the present disclosure.

FIGS. 8D and 8E exemplarily show the change in carbon-carbon double bond conversion as a function of PGDA weight content in the PGDA-PAA SME and UV exposure time, respectively, in accordance with various embodiments of the present disclosure.

FIG. 9 exemplarily shows DSC curves of the PGDA-PAA SME samples with different PGDA weight ratios of 50 wt %, 60 wt %, and 70 wt % in accordance with various embodiments of the present disclosure.

FIGS. 10A and 10B exemplarily show the changes in tensile strength of the biocompatible SME as a function of the relative weight percentage of PGDA below the transition temperature and above the transition temperature, respectively, in accordance with various embodiments of the present disclosure.

FIG. 10C exemplarily shows the changes in tensile strength of the biocompatible SME as a function of the number of cyclic tensile tests performed, in accordance with various embodiments of the present disclosure.

FIG. 11 exemplarily shows the changes in Young's modulus and toughness in the biocompatible SME as functions of the PGDA weight ratio, in accordance with various embodiments of the present disclosure when tested below the transition temperature and above the transition temperature.

FIG. 12A exemplarily shows the temperature-dependent storage modulus and ratio of loss modulus as a function of the temperature of the biocompatible SME, in accordance with various embodiments of the present disclosure.

FIG. 12B exemplarily shows the change in shape fixity ratio, R_{f}, and the shape recovery ratio, Rᵣ, as a function of the PGDA weight ratio in the biocompatible SME, in accordance with various embodiments of the present disclosure.

FIG. 13 exemplarily depicts the biodegradability of the biocompatible SME as a function of the PGDA weight ratios in the biocompatible SME as well as the solvent used, in accordance with various embodiments of the present disclosure.

FIGS. 14A and 14B exemplarily show the biocompatibility of the biocompatible SME by depicting the cell count and survival rate of cells in contact with the biocompatible SME, respectively, in accordance with various embodiments of the present disclosure.

FIGS. 15A-15B exemplarily show differences in the swelling ratio of hydrated PGDA-PUA-PAA over time as a function of the PGDA:PUA:PAA weight ratios and as a function of the hydration solvent/temperature mixture, respectively, in accordance with various embodiments of the present disclosure.

FIG. 16 exemplarily shows changes in the first transition temperature and the second transition temperature of exemplary PGDA-PUA-PAA networks, when hydrated and when dry, as a function of the weight percentage of PGDA in each PGDA-PUA-PAA network, in accordance with various embodiments of the present disclosure.

FIG. 17A exemplarily shows changes in the shape fixity ratio and shape recovery ratio of an exemplary PGDA-PUA-PAA SME as a function of SME thickness, in accordance with various embodiments of the present disclosure.

FIG. 17B exemplarily shows changes in the shape recovery ratio of an exemplary PGDA-PUA-PAA multi-transition SME as a function of time and temperature, in accordance with various embodiments of the present disclosure.

FIG. 18A exemplarily provides microscopy images of the separation of phases in the PGDA-PUA-PAA multi-transition SME during hydration as a function of time, in accordance with various embodiments of the present disclosure.

FIG. 18B exemplarily provides spatiotemporal intensity difference maps and related histograms demonstrating the separation of phases in the PGDA-PUA-PAA multi-transition SME during hydration as a function of time, in accordance with various embodiments of the present disclosure.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what is believed to be the best mode of carrying out the claimed invention. Additionally, it is to be understood that the claimed invention is not limited in its applications to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

The term “polymer” as used herein is considered to be inclusive of polymers made from a single repeating monomeric subunit as well as what are commonly called “copolymers,” or polymers made from more than one monomeric subunit. The term “copolymer” is used herein specifically to denote polymers made from more than one type of repeating monomeric subunit. The term “elastomer” is a kind of polymer showing high elasticity and stretchability.

The terms “biocompatible” and “biocompatibility” as used herein refer to a material's ability to perform its intended function within a biological system, such as the human body, without eliciting undesirable local or systemic responses such as cell death, impaired cell function, or immune response.

The term “biodegradable” as used herein refers to materials capable of being decomposed by natural biological processes into simpler compounds through the action of human bodies and/or their enzymes under physiological conditions.

The term “transition temperature” as used herein refers to the critical temperature at which a shape memory material undergoes a phase change that enables it to switch between relatively rigid and flexible states.

The term “programmed shape” as used herein refers to the shape that a shape memory material is physically manipulated into holding. Shape memory materials are typically created with an ‘original’ shape, and programming typically involves deforming the material's original shape at elevated temperatures (above a transition temperature) to create a temporary shape that is then fixed by cooling below a transition temperature. The original shape essentially remains stored in the material's molecular structure. When the material is in a programmed shape and is then heated above a transition temperature, the original shape is restored. As detailed below, some materials can have multiple transition temperatures and thus multiple programmed shapes. For example, a material holding a second programmed shape, when heated above a first transition temperature, will assume a first programmed shape, and when further heated above a second transition temperature, will assume its original shape.

The term “shape fixity” as used herein refers to a measure of a material's ability to maintain its programmed temporary shape after any deforming forces are removed and the material is cooled below its transition temperature, typically expressed as a percentage ratio (shape fixity ratio, R_{f}) between the achieved temporary shape and the intended programmed shape.

The term “shape recovery” as used herein refers to a measure of a material's ability to return to its original shape from its temporary shape when heated above its transition temperature, expressed as a percentage (shape recovery ratio, Rᵣ) of the total deformation that is reversed during recovery. The shape recovery of the exemplary SMEs described herein is triggered by internal body heat, but can be done by any means known to one of ordinary skill in the art, including by external magnetic and optical triggers if they form composites.

The term “hydrogel” as used herein refers to a three-dimensional network of polymer and/or copolymer chains that can absorb and retain significant amounts of water while maintaining their structural integrity.

The term “crosslinking density” as used herein refers to the number of crosslinks (covalent or non-covalent bonds) connecting different polymer chains per unit volume in a polymer network, which determines the average molecular weight between these interconnections and directly influences the material's mechanical properties, swelling behavior, and degradation rate.

The term “hydrophobic character” as used herein refers to the tendency of a material or molecular region to repel or minimize interactions with water molecules, typically due to the presence of non-polar groups such as alkyl chains, aromatic rings, or other low-polarity molecular segments.

Described herein are shape memory elastomers (SMEs) that exhibit tunable mechanical properties, high elasticity and stretchability, transition temperatures that can correspond to human physiological conditions, high shape fixity, rapid recovery from their programmed shapes to their original shapes, high biocompatibility and biodegradability. In some embodiments, the SMEs described herein also exhibit multiple transition temperatures, multiple shape changes, and a controlled onset duration for transition between a programmed shape and the original shape. Thus, the SMEs described herein are ideal for use as biomedical implants, and, as is further detailed below, enable unique and powerful opportunities for minimally-invasive microsurgeries. Methods of manufacture for the described SMEs are also provided herein.

The following detailed description begins with description of a broad general embodiment of the SMEs described herein that principally comprises a copolymer with two polymer components, referred to as a biocompatible SME, followed by a description of embodiments that comprise a third polymer component, referred to as a multi-transition SME. Methods of preparation for each of the biocompatible SME and the multi-transition SME are provided as well, as are descriptions of biomedical implants based on the SMEs described herein.

### Biocompatible SME

The present disclosure generally provides a biocompatible shape memory elastomer (SME) 50 that exhibits high ductility, elasticity, and mechanical strength, and features a single transition temperature that can be set at or near internal human body temperature. In various exemplary embodiments, the biocompatible SME 50 is a copolymer comprising a first polymer and a second polymer that are mutually crosslinked to form a copolymer network. The first polymer is a biocompatible, biodegradable shape memory polymer with hydrophobic character and the second polymer is a biocompatible, biodegradable hydrophilic polymer that has one or more functional groups capable of forming hydrogen bonds. In various exemplary embodiments, the hydrogen bonds formed by the second polymer increase the cross-linking density of the copolymer network.

In various exemplary embodiments, the first polymer is poly(glycerol dodecanoate acrylate) (PGDA) and the second polymer is polyacrylic acid (PAA), which mutually crosslink to form the copolymer network, which is a PGDA-PAA network 100. Molecular structures of PGDA and PAA are depicted below as Structure 1 and Structure 2, respectively, where n and n′ denote that each polymer comprises arbitrary numbers of monomeric subunits.

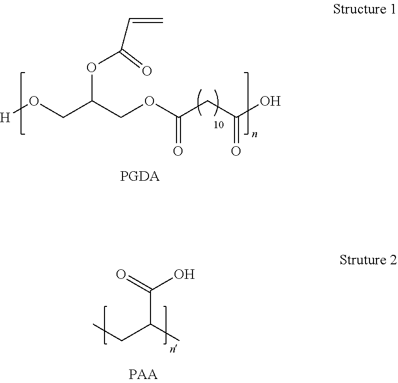

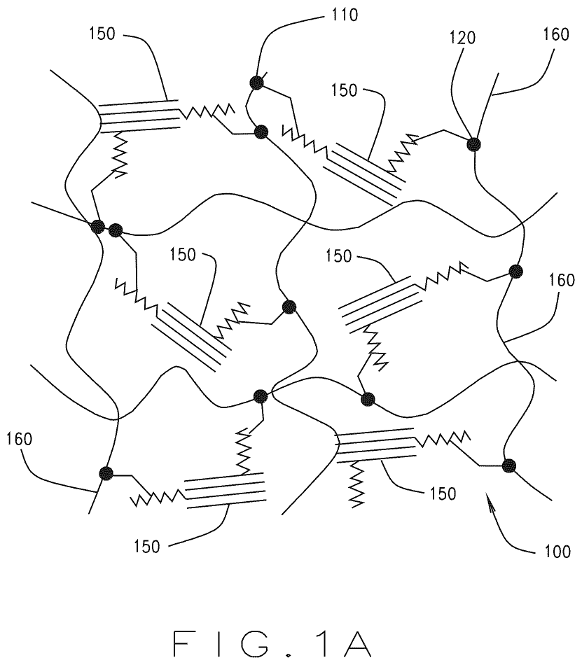

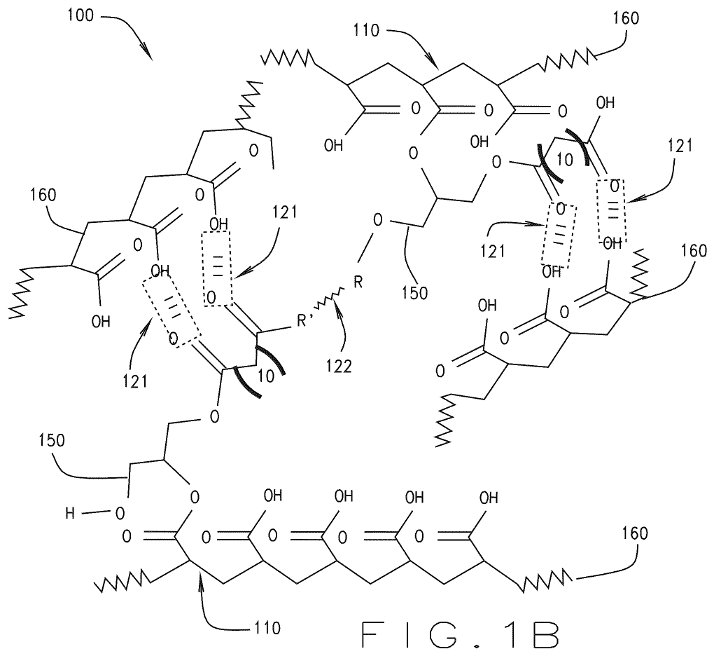

FIGS. 1A and 1B depict exemplary schematics of the PGDA-PAA network 100 which defines the physical and chemical properties of the biocompatible SME 50. Note that FIGS. 1A and 1B only show representative segments of the molecular structure of the PGDA-PAA network 100. Unlike crystalline materials which have a precise, repeating arrangement, the actual structure of the PGDA-PAA network 100 varies throughout the biocompatible SME 50. FIGS. 1A and 1B illustrate typical bonding patterns and molecular arrangements that occur within the PGDA-PAA network 100 but should not be interpreted as depicting the exact structure found at every point in the biocompatible SME 50. As can be appreciated from FIG. 1A, the PGDA-PAA network 100 comprises a plurality of PGDA moieties 150 and a plurality of PAA moieties 160 connected to one another by both covalent bonds 110 and non-covalent interactions 120. An exemplary molecular-level structure of the PGDA-PAA network 100 is provided in further detail in FIG. 1B, which shows PGDA moieties 150 connected to PAA moieties 160 through covalent bonds 110, PGDA moieties 150 connected to PAA moieties 160 by non-covalent hydrogen bonds 121, and a PGDA moiety 150 connected to another PGDA moiety 150 by non-covalent dispersion forces 122. The resulting PGDA-PAA network 100 is thus highly interconnected, but also characterized by somewhat distinct domains of PGDA moieties 150 and PAA moieties 160.

In various exemplary embodiments, the PGDA-PAA network 100 comprises 70% PGDA by weight and 30% PAA by weight, although a wide range of other ratios of PGDA:PAA are considered to be within the scope of the present disclosure and are explored in further detail below in the Examples. In various exemplary embodiments, the weight ratio of PGDA:PAA in the PGDA-PAA network 100 can be between 99.99:0.01 and 0.01:99.99, including all ratios to a precision of 0.01, as even very small variations in the percentages of PGDA and PAA can significantly alter the properties of the PGDA-PAA network 100. In various exemplary embodiments, the weight ratio of PGDA:PAA in the PGDA-PAA network 100 can be 99.99:0.01, 99.9:0.1, 99:1, 98:2, 97:3, 96:4, 95:5, 94:6, 93:7, 92:8, 91:9, 90:10, 89:11, 88:12, 87:13, 86:14, 85:15, 84:16, 83:17, 82:18, 81:19, 80:20, 79:21, 78:22, 77:23, 76:24, 75:25, 74:26, 73:27, 72:28, 71:29, 70:30, 69:31, 68:32, 67:33, 66:34, 65:35, 64:36, 63:37, 62:38, 61:39, 60:40, 59:41, 58:42, 57:43, 56:44, 55:45, 54:46, 53:47, 52:48, 51:49, 50:50, 49:51, 48:52, 47:53, 46:54, 45:55, 44:56, 43:57, 42:58, 41:59, 40:60, 39:61, 38:62, 37:63, 36:64, 35:65, 34:66, 33:67, 32:68, 31:69, 30:70, 29:71, 28:72, 27:73, 26:74, 25:75, 24:76, 23:77, 22:78, 21:79, 20:80, 19:81, 18:82, 17:83, 16:84, 15:85, 14:86, 13:87, 12:88, 11:89, 10:90, 9:91, 8:92, 7:93, 6:94, 5:95, 4:96, 3:97, 2:98, 1:99, 0.1:99.9, or 0.01:99.99.

The exemplary ratio provided above of 70% PGDA to 30% PAA by weight is best understood in recognition of how PGDA and PAA each affect the physical and chemical properties of the PGDA-PAA network 100. The following description therefore details how PGDA and PAA each affect the shape memory behavior, thermomechanical properties, and biocompatibility of the PGDA-PAA network 100. With this information, the particular properties of the biocompatible SME 50 described herein can be tailored to a given application.

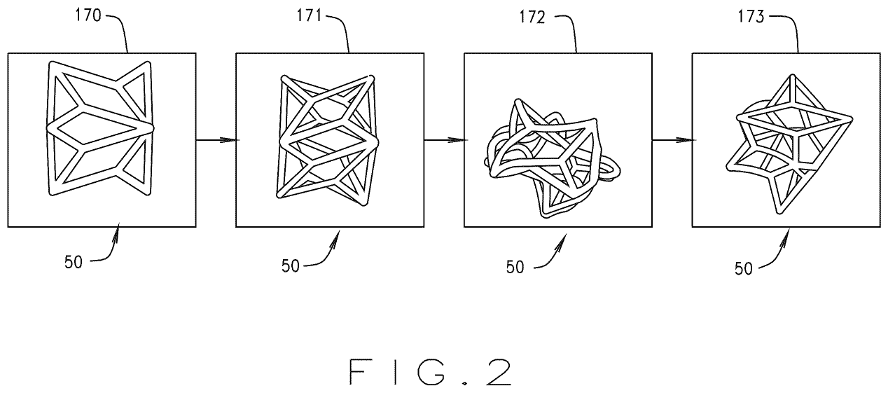

Without being bound by any particular theory, it is believed that the primary role of PGDA in the PGDA-PAA network 100 is to impart the shape memory behavior, while the primary role of PAA is to impart ductility, elasticity and mechanical strength. An example of the shape memory behavior of biocompatible SME 50 of the present disclosure is provided in FIG. 2. As can be seen in the exemplary depiction of FIG. 2, the biocompatible SME 50 is manufactured in the original shape 170. Although the original shape 170 depicted in FIG. 2 is a wireframe of a polyhedron, the original shape 170 can assume a nearly infinite variety of forms, as the original shape 170 is set in the process of manufacturing the biocompatible SME 50 itself. When the original shape 170 of the biocompatible SME 50 comprising the PGDA-PAA network 100 is heated above a transition temperature Tₜᵣₐₙₛ, the more rigid, crystalline domains of PGDA undergo a physical change to form an amorphous, homogenous phase with high mobility, thereby rendering the entire PGDA-PAA network 100 more susceptible to deformation. The susceptibility of the PGDA-PAA network 100 to deformation above Tₜᵣₐₙₛ is visible in the less rigid shape 171 of the biocompatible SME 50. Deformation of the biocompatible SME 50 above Tₜᵣₐₙₛ and a subsequent cooling of the biocompatible SME 50 to room temperature (below Tₜᵣₐₙₛ) causes the PGDA domains to solidify and fix the biocompatible SME 50 in a programmed shape 172. In order to recover the original shape, or at least a shape that is similar to the original shape, the biocompatible SME 50 can then undergo heating again to a temperature above Tₜᵣₐₙₛ, which causes the PGDA moieties to once again acquire a more amorphous character, and the biocompatible SME 50 thereby forms a restored shape 173. The restored shape 173 can be an exact match for the original shape 170, or it can be an approximate match. In various exemplary embodiments, the restored shape 173 is a sufficient match for the original shape 170 for a user's purpose. As PGDA imparts the shape memory behavior into the PGDA-PAA network 100, the extent to which the restored shape 173 is a match for the original shape 170 is strongly influenced by the relative proportion of PGDA in the PGDA-PAA network 100. Thus, in various exemplary embodiments, a greater proportion of PGDA than PAA by weight in the PGDA-PAA network 100 such as the 70% PGDA to 30% PAA by weight ratio cited above helps the restored shape 173 more closely match the original shape 170. This effectively means that higher proportions of PGDA in the PGDA-PAA network enable the biocompatible SME 50 to exhibit stronger, more repeatable shape memory behavior.

The effect of PGDA on the shape memory function of the biocompatible SME 50 manifests in the shape recovery ratio (Rᵣ) and the shape fixity ratio (R_{f}). The shape recovery ratio declines slightly as the PGDA ratio in the PGDA-PAA network 100 decreases from 70%, while the shape fixity ratio decreases dramatically as the PGDA ratio in the PGDA-PAA network 100 decreases from 70%, as further detailed below in Example 14. Thus, in various exemplary embodiments, maintaining a higher proportion of PGDA than PAA by weight in the PGDA-PAA network 100 helps the biocompatible SME 50 to fully recover the original shape after being heated above the transition temperature.

The effect of PAA on altering the ductility and tensile strength of the PGDA-PAA network 100 in the biocompatible SME 50 manifests in the transition temperature Tₜᵣₐₙₛ, as well as the measured elasticity and toughness of the biocompatible SME 50. For example, a change in the ratio of PGDA:PAA from 70:30 to 50:50 results in a decrease in Tₜᵣₐₙₛ of approximately eight degrees Celsius. Thus, higher concentrations of PAA in the PGDA-PAA network 100 result in higher Tₜᵣₐₙₛ values. Without being bound by any particular theory, PAA's effect on the transition temperature Tₜᵣₐₙₛ is thought to be attributable to PAA's ability to change the cross-linking density of the PGDA-PAA network 100. As shown in FIG. 1B, the PAA moieties 150 can form covalent bonds with the PGDA moieties 160, and the PAA moieties 150 also comprise hydroxyl functional groups that can form noncovalent hydrogen bonds with the PGDA moieties 160. By decreasing the relative concentration of PAA in the PGDA-PAA network 100, one reduces the number of covalent bonds and noncovalent interactions in the PGDA-PAA network 100, thereby decreasing its crosslinking density and thus lowering the transition temperature Tₜᵣₐₙₛ. Thus, the transition temperature Tₜᵣₐₙₛ in the biocompatible SME 50 can be tuned to meet the needs of a given application, in part, by adjusting the relative concentration of PAA in the PGDA-PAA network 100.

The increase in crosslinking density in the PGDA-PAA network 100 that results from increasing the relative concentration of PAA also causes the resulting biocompatible SME 50 to have a higher tensile strength. For example, in various embodiments, changing the PGDA:PAA weight ratio from 70:30 to 55:45 nearly doubles the tensile strength of the biocompatible SME 50 at room temperature (below the transition temperature Tₜᵣₐₙₛ). Similarly, the Young's modulus (E) and toughness (U_{T}) also decrease as the relative concentration of PAA in the PGDA-PAA network 100 is decreased. Increased PAA concentrations also result in the biocompatible SME 50 having greater elasticity, as measured by the strain at failure ε_{f}. As further detailed below in Example 13, the strain at failure ε_{f} values (measured above and below the transition temperature Tₜᵣₐₙₛ) show modest declines as the relative concentration of PAA in the PGDA-PAA network 100 is decreased. Thus, mechanical properties such as the tensile strength, Young's modulus, toughness, and elasticity of the biocompatible SME 50 can be tuned to meet the needs of a given application, in part, by adjusting the relative concentration of PAA in the PGDA-PAA network 100. Such tuning enables the biocompatible SME 50 to be more easily and safely deployed in vivo as a biomedical implant, as the physical and mechanical properties of the biocompatible SME 50 can be adjusted to better emulate the characteristics of the natural tissues into which such a biomedical implant would be embedded.

Both PGDA and PAA are strongly biocompatible and thus show little to no evidence of detrimental effects on living tissue, as further described below in Example 15. PGDA is also significantly biodegradable, which is important for any biomedical implant application, as upon implantation, the biocompatible SME 50 comprising the PGDA-PAA network 100 is expected to provide temporary support for cell growth until it is replaced by regenerated tissue.

One of ordinary skill in the art could envision modifications to the above description of the biocompatible SME 50 that are considered to be within the scope of the present description. For example, in lieu of PGDA, the first polymer could be any other polymer that exhibits shape memory function, including but not limited to polyurethane, polycaprolactone, polylactic acid, polyethylene, polystyrene, polynorborene, polymethyl methacrylate, polyvinyl chloride, poly(ethylene-co-vinyl acetate), and polyimides. Polycaprolactone, polylactic acid, polyethylene glycol, polyethylene glycol diacrylate, and alginate in particular are believed to have significant biodegradability, and thus these and similarly biodegradable materials are held to be within the scope of the present disclosure. Similarly, in lieu of PAA, one can envision using other polymer components that improve the extent of crosslinking through increased covalent bonding, increased noncovalent interactions, or both, including but not limited to polyvinyl alcohol, polyethylene glycol, polyethylene glycol diacrylate, polyacrylamide, polymethacrylic acid, hyaluronic acid, chitosan, tannic acid, polyitaconic acid, maleic acid, and other biomaterials like alginate, peptide, chitosan, and collagen. Note that in the above exemplary lists of materials, functional groups such as carboxylic acids, hydroxyls, amides, and phenols are present to enable hydrogen bonding and/or increased covalent bonding to promote crosslinking.

### Biocompatible SME Preparation

An exemplary biocompatible SME 50 comprising the PGDA-PAA network 100 can be synthesized according to a three-dimensional (3D) printing process. First, a pre-polymer synthesis is conducted by combining polyol and dicarboxylic acid components in an equimolar ratio. This mixture undergoes thermal polymerization at elevated temperature under inert gas conditions with continuous agitation, resulting in the pre-polymer or oligomer. Second, the resulting pre-polymer is functionalized through an acrylation process. This involves preparing a base solution containing a polymerization inhibitor, a catalyst, a base, and a solvent. In various exemplary embodiments, the polymerization inhibitor is 4-methoxyphenol, the catalyst is 4-dimethylaminopyridine, the base is triethylamine, and the solvent is dichloromethane. The pre-polymer is dissolved in the base solution to form a pre-polymer solution, which is cooled to low temperature under inert conditions. For example, in various exemplary embodiments, the pre-polymer solution is cooled to 0° C. under nitrogen atmosphere. Acryloyl halide, diluted in the solvent, is then added dropwise to achieve the desired degree of functionalization, resulting in a functionalized pre-polymer. In various exemplary embodiments, the functionalized pre-polymer is pre-PGDA.

Third, the pre-polymer solution is maintained at ambient temperature with protection from light. In various exemplary embodiments, after a specified reaction period, more of the polymerization inhibitor can be introduced. The functionalized pre-polymer is then isolated. In various exemplary embodiments, the functionalized pre-polymer can be isolated according to any means known to one of ordinary skill of the art, including but not limited to the following series of steps: solvent removal followed by dissolution in an organic ester solvent, drying under vacuum, and centrifugation to remove reaction byproducts.

The intrinsic shape recovery of the exemplary PGDA-PAA network 100 is triggered by body heat. To make the external trigger possible, additives such as magnetically and optically active particles in forms of nanoparticles and microparticles can be added into the PGDA-PAA network 100 to form a composite.

A printing resin is prepared by first making an initial resin mixture, which is done by combining the functionalized pre-polymer with a first monomer. In various exemplary embodiments, the first monomer is acrylic acid. In various exemplary embodiments, the initial resin mixture comprises 50-70% functionalized pre-polymer by weight. A photoinitiator is added to the initial resin mixture, resulting in a homogenous resin. In various exemplary embodiments, the photoinitiator is diphenyl(2,4,6-trimethylbenzoyl) phosphine oxide (TPO). The homogeneous resin is then used to print the biocompatible SME 50 using digital light processing (DLP) printing technology in accordance with techniques known to one of ordinary skill in the art or other 3D printing techniques such as stereolithography, extrusion-based 3D printing. For example, the DLP printing can, in various exemplary embodiments, be performed with an irradiation wavelength of 405 nm, a power density of approximately 5 mW/cm², and with a layer thickness setting of 50 μm. In various exemplary embodiments, the biocompatible SME 50 can undergo post-processing steps as known to one of ordinary skill in the art, including removal from the build platform, washing with ethanol to eliminate unreacted components, and photo-curing to complete the crosslinking process. Additionally, other manufacturing methods can be envisioned as known to one of ordinary skill in the art, including molding, extrusion, and machining. Further exemplary 3D printing methods include extrusion-based 3D printing (direct ink printing), and light-based 3D printing (Digital Light Processing, DLP, and Liquid Crystal Display, LCD).

### Multi-Transition SME

In various exemplary embodiments, one can further bolster the biocompatible SME 50 with one or more additional components in order to achieve multiple transition temperatures, multiple shape changes, and a controlled onset duration for transition between a programmed shape and the original shape, referred to herein as the multi-transition SME 60. The inclusion of additional materials such as poly(10-undecenoic acid) (PUA) in the above-described SMEs can create a network that comprises a three-component network. The three-component network comprises the first polymer, the second polymer, and a third polymer. The third polymer is a biodegradable, biocompatible shape memory polymer with hydrophobic character. An exemplary SME comprising the three-component network, herein called the multi-transition SME 60, comprises PGDA as the first polymer, PAA as the second polymer and PUA as the third polymer, and exhibits additional beneficial features over the biocompatible SME 50 as described below.

The multi-transition SME 60 comprises PGDA, PAA, and PUA, with PUA having the molecular structure shown below in Structure 3. PUA is polymerized from 10-undecenoic acid (UA), and is considered to be a biodegradable, flexible polymer that exhibits thermoplastic properties, meaning that it softens on heating and hardens on cooling.

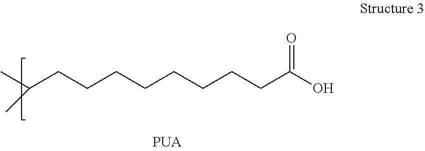

In various exemplary embodiments, the PGDA, PAA, and PUA are mutually crosslinked to form a PGDA-PUA-PAA network.

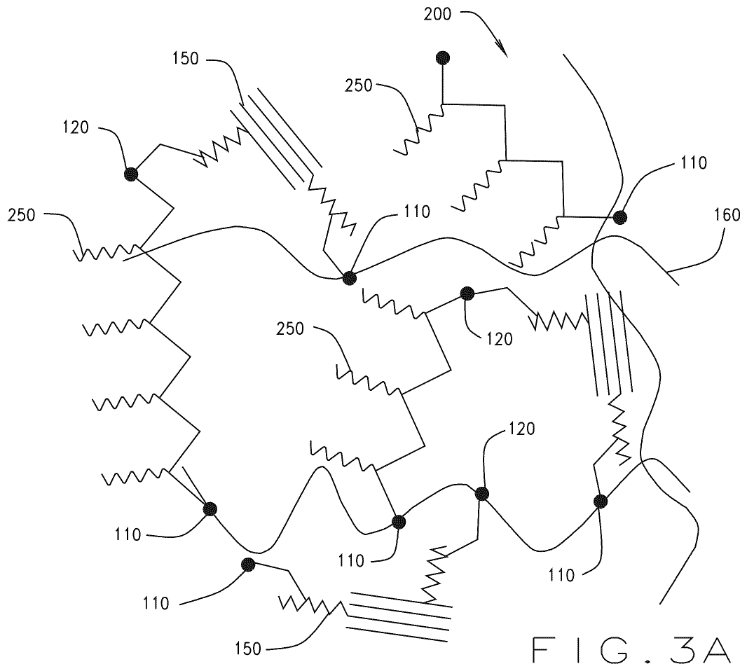

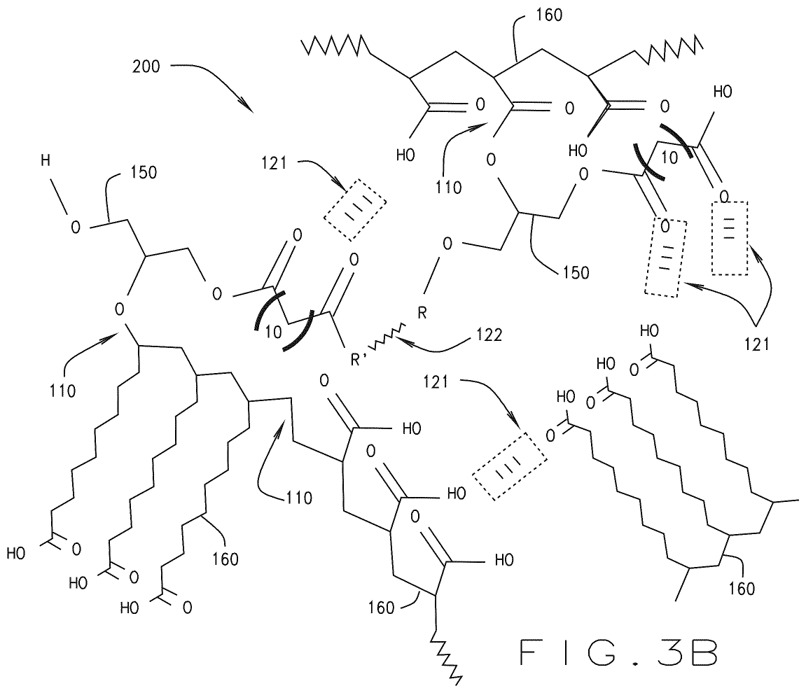

FIGS. 3A and 3B depict exemplary schematics of a PGDA-PUA-PAA network 200. Note that FIGS. 3A and 3B only show representative segments of the molecular structure of the PGDA-PUA-PAA network 200. Unlike crystalline materials which have a precise, repeating arrangement, the actual structure of the PGDA-PUA-PAA network 200 varies throughout the material. FIGS. 3A and 3B illustrate typical bonding patterns and molecular arrangements that occur within the PGDA-PUA-PAA network 200, but should not be interpreted as depicting the exact structure found at every point in the material. As can be appreciated from FIG. 3A, the PGDA-PUA-PAA network 200 comprises a plurality of PGDA moieties 150, a plurality of PAA moieties 160, and a plurality of PUA moieties 250 connected to one another by both covalent bonds 110 and non-covalent interactions 120. An exemplary molecular-level structure of the PGDA-PUA-PAA network 200 is provided in further detail in FIG. 3B, which shows PUA moieties 250 bonded to PGDA moieties 150 through covalent bonds 110, PUA moieties 250 bonded to PAA moieties 150 through covalent bonds 110, PUA moieties 250 bonded to PGDA moieties 150 through non-covalent hydrogen bonds 121, PUA moieties 250 bonded to PAA moieties 150 through non-covalent hydrogen bonds 121, PGDA moieties 150 bonded to PAA moieties 160 through covalent bonds 110, PGDA moieties 150 bonded to PAA moieties 160 by non-covalent hydrogen bonds 121. The resulting PGDA-PUA-PAA network 200 is thus highly interconnected, much like the PGDA-PAA network 100, albeit with a more complex variety of covalent and noncovalent interactions.

In various exemplary embodiments, the PGDA-PUA-PAA network 200 comprises 50% PGDA by weight, 20% PUA by weight, and 30% PAA by weight, although a wide range of other ratios of PGDA:PUA:PAA are considered to be within the scope of the present disclosure and are explored in further detail below in the Examples. In various exemplary embodiments, the PGDA-PUA-PAA network can comprise between 99.99% and 0.01% PGDA, between 99.99% and 0.01% PUA, and between 99.99% and 0.01% PAA, so long as all three numbers sum to 100%. In various exemplary embodiments, the weight ratio of PGDA:PUA:PAA in the PGDA-PUA-PAA network 200 can be exactly or approximately 99:0.5:0.5, 98:1:1, 95:2.5:2.5, 90:5:5, 85:5:10, 85:10:5, 80:5:15, 80:10:10, 80:15:5, 75:5:20, 75:10:15, 75:15:10, 75:20:5, 70:5:25, 70:10:20, 70:15:15, 70:20:10, 70:25:5, 65:5:30, 65:10:25, 65:15:20, 65:20:15, 65:25:10, 65:30:5, 60:5:35, 60:10:30, 60:15:25, 60:20:20, 60:25:15, 60:30:10, 60:35:5, 55:5:40, 55:10:35, 55:15:30, 55:20:25, 55:25:20, 55:30:15, 55:35:10, 55:40:5, 50:5:45, 50:10:40, 50:15:35, 50:20:30, 50:25:25, 50:30:20, 50:35:15, 50:40:10, 50:45:5, 45:5:50, 45:10:45, 45:15:40, 45:20:35, 45:25:30, 45:30:25, 45:35:20, 45:40:15, 45:45:10, 45:50:5, 40:5:55, 40:10:50, 40:15:45, 40:20:40, 40:25:35, 40:30:30, 40:35:25, 40:40:20, 40:45:15, 40:50:10, 40:55:5, 35:5:60, 35:10:55, 35:15:50, 35:20:45, 35:25:40, 35:30:35, 35:35:30, 35:40:25, 35:45:20, 35:50:15, 35:55:10, 35:60:5, 30:5:65, 30:10:60, 30:15:55, 30:20:50, 30:25:45, 30:30:40, 30:35:35, 30:40:30, 30:45:25, 30:50:20, 30:55:15, 30:60:10, 30:65:5, 25:5:70, 25:10:65, 25:15:60, 25:20:55, 25:25:50, 25:30:45, 25:35:40, 25:40:35, 25:45:30, 25:50:25, 25:55:20, 25:60:15, 25:65:10, 25:70:5, 20:5:75, 20:10:70, 20:15:65, 20:20:60, 20:25:55, 20:30:50, 20:35:45, 20:40:40, 20:45:35, 20:50:30, 20:55:25, 20:60:20, 20:65:15, 20:70:10, 20:75:5, 15:5:80, 15:10:75, 15:15:70, 15:20:65, 15:25:60, 15:30:55, 15:35:50, 15:40:45, 15:45:40, 15:50:35, 15:55:30, 15:60:25, 15:65:20, 15:70:15, 15:75:10, 15:80:5, 10:5:85, 10:10:80, 10:15:75, 10:20:70, 10:25:65, 10:30:60, 10:35:55, 10:40:50, 10:45:45, 10:50:40, 10:55:35, 10:60:30, 10:65:25, 10:70:20, 10:75:15, 10:80:10, 10:85:5, 5:5:90, 5:10:85, 5:15:80, 5:20:75, 5:25:70, 5:30:65, 5:35:60, 5:40:55, 5:45:50, 5:50:45, 5:55:40, 5:60:35, 5:65:30, 5:70:25, 5:75:20, 5:80:15, 5:85:10, 5:90:5, 2.5:95:2.5, 1:98:1, 0.5:99:0.5, 5:5:90, 2.5:2.5:95, 1:1:98, and 0.5:0.5:99.

The exemplary ratio provided above of 50% PGDA by weight, 20% PUA by weight, and 30% PAA by weight is best understood in recognition of how the introduction of PUA makes the PGDA-PUA-PAA network 200 physically and chemically different from the PGDA-PAA network 100. Without being bound by any particular theory, PGDA and PAA appear to have largely the same effects on the PGDA-PUA-PAA network 200 as they have on the PGDA-PAA network 100 as described above. The following description therefore details how PUA in particular affects the shape memory behavior, thermomechanical properties, and biocompatibility of the PGDA-PUA-PAA network 200. With this information, the particular properties of the multi-transition SME 60 described herein can be tailored to a given application.

The multi-transition SME 60, by virtue of the inclusion of PUA, features two transition temperatures: a first transition temperature T₁ and a second transition temperature T₂ that is higher than the first transition temperature. Without being bound by any particular theory, it is believed that the primary role of PUA in the PGDA-PUA-PAA network 200 is to impart additional shape memory behavior and thereby introduce the first transition temperature, while the second transition temperature is imparted by the PGDA. The inclusion of PUA is also believed to impart a controlled onset for shape change behavior at the second transition temperature, and impart mechanical properties that enable the multi-transition SME 60 to assume hydrogel-like qualities. The exact value of the first transition temperature T₁ is dependent on the ratio of PGDA:PUA:PAA as well as the level of hydration of the multi-transition SME 60, the latter of which is a phenomenon that is explored further below.

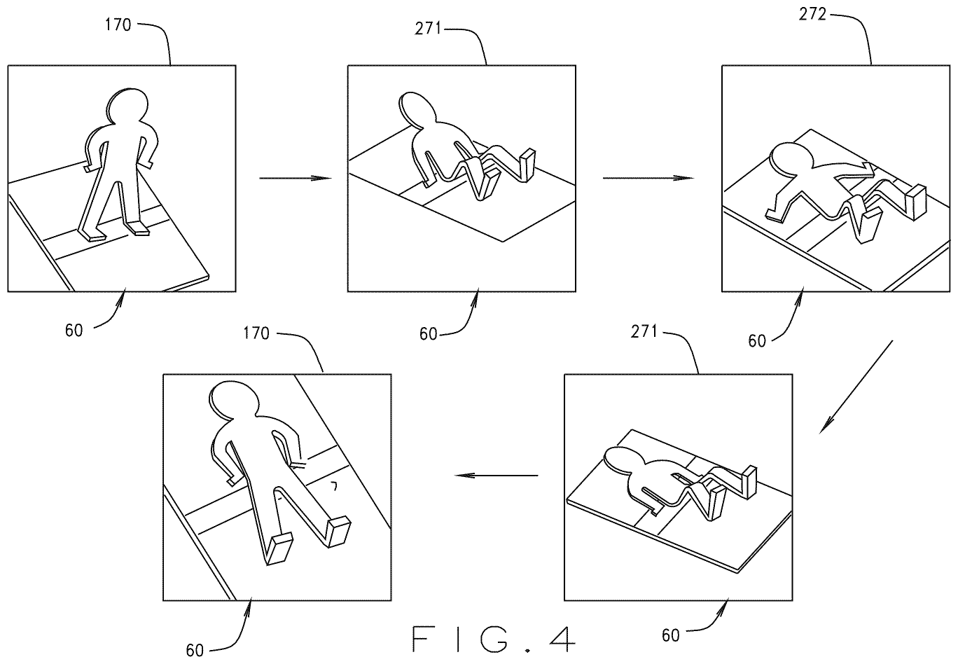

Thus, the multi-transition SME 60, by virtue of having two transition temperatures, can also have four shapes: the original shape, a first programmed shape, a second programmed shape, and a final swelled shape after water uptake. These four shapes and their relationship to the two transition temperatures are elucidated by the exemplary depiction of FIG. 4. The original shape 170 is the shape in which the multi-transition SME 60 was originally manufactured, which, in the exemplary depiction of FIG. 4, is a figure of a stick figure standing up. The multi-transition SME 60 can then undergo heating above the second transition temperature, T₂, which renders the multi-transition SME 60 susceptible to deformation due to the softening of PGDA moieties. In the exemplary depiction of FIG. 4, the multi-transition SME 60 is deformed to assume the shape of a stick figure sitting down with its arms at its side, and then cooled to a temperature between the second transition temperature T₂ and the first transition temperature T₁, resulting in the first programmed shape 271. Between the second transition temperature T₂ and the first transition temperature T₁, the PGDA moieties are relatively crystallized, but the PUA domains are not, leaving the multi-transition SME 60 still somewhat susceptible to deformation. Thus, in the exemplary depiction of FIG. 4, the multi-transition SME 60 is further deformed to assume the shape of a stick figure laying down with its arms extended, and then cooled to below the first transition temperature T₁, resulting in the second programmed shape 272. Below the first transition temperature T₁, both the PGDA moieties and the PUA moieties are relatively crystallized, and the multi-transition SME 60 is no longer susceptible to deformation. In order to restore the original shape, the multi-transition SME 60 can then be heated again. After the temperature of the multi-transition SME 60 is brought above the first transition temperature T₁, the first programmed shape 271 begins to be restored, resulting in the stick figure reassuming a seated position with its arms at its side. Ultimately, when the temperature of the multi-transition SME 60 is brought above the second transition temperature T₂, the original shape 170 is restored, resulting in the stick figure once again in a standing pose.

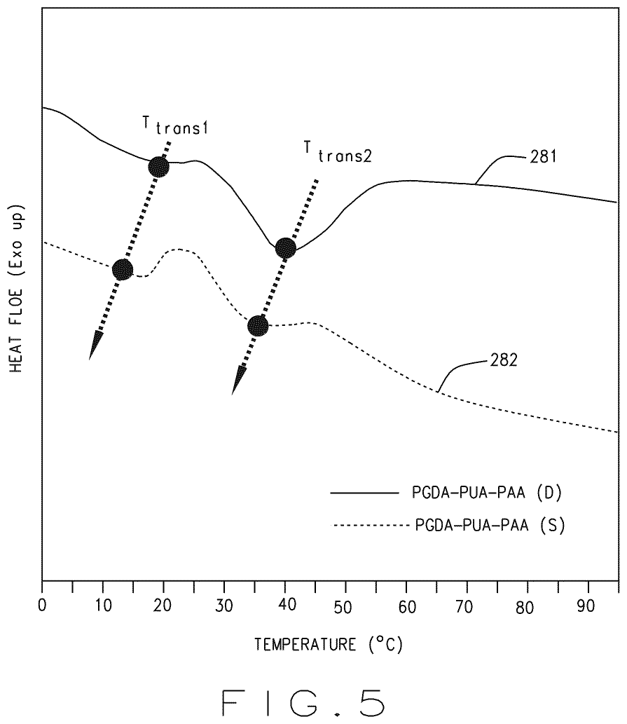

The incorporation of PUA into the PGDA-PUA-PAA network 200 also manifests in the multi-transition SME 60 exhibiting a unique response to being hydrated: both of the two transition temperatures decrease slightly, which enables a controlled onset for shape change behavior occurs at the second transition temperature T₂. The change in each of the two transition temperatures is depicted in FIG. 5, which is a differential scanning calorimetry curve that shows how the first transition temperature T₁ and the second transition temperature T₂ both differ between an exemplary dry PGDA-PUA-PAA network 281 and an exemplary hydrated PGDA-PUA-PAA network 282. In various exemplary embodiments, the change in the first transition temperature T₁ between the exemplary dry PGDA-PUA-PAA network 281 and the exemplary hydrated PGDA-PUA-PAA network 282 is a decrease of approximately 3° C., and the change in the second transition temperature T₂ between the exemplary dry PGDA-PUA-PAA network 281 and the exemplary hydrated PGDA-PUA-PAA network 282 is a decrease of approximately 6° C.

The decrease in the second transition temperature T₂ when the PGDA-PUA-PAA network 200 is hydrated enables a controlled onset for shape change behavior at the second transition temperature T₂. As an example, consider a multi-transition SME 60 that, in various exemplary embodiments, has a PGDA-PUA-PAA network 200 with a PGDA:PUA:PAA weight ratio of 50:20:30 and that has a second transition temperature T₂ that is 40° C. when dry and 34° C. when fully hydrated. If placed in an environment at approximately 34° C. when dry, the exemplary multi-transition SME 60 will not undergo a shape change transition to its original shape because the environmental temperature is below that of the dry second transition temperature T₂ (40° C.). However, if the exemplary multi-transition SME 60 is then exposed to water or a similar solvent, the multi-transition SME 60 will hydrate. As the multi-transition SME 60 hydrates, its second transition temperature will gradually decrease until the multi-transition SME 60 is fully hydrated (the final swelled shape), at which point the second transition temperature T₂ is 34° C. Since the second transition temperature T₂ of the exemplary fully-hydrated multi-transition SME 60 is at this point equal to the applied temperature of 34° C., the multi-transition SME 60 will then undergo a shape change transition to its original shape. Thus, it can be seen that for the multi-transition SME 60, the transition between the first programmed shape and the original shape at the second transition temperature T₂ can, in various exemplary embodiments, be a multi-activation process comprising a thermal activation (applying at least a minimum temperature) and a fluid activation (applying a level of hydration). The time required for the multi-transition SME 60 to hydrate sufficiently to bring its second transition temperature T₂ down to the applied temperature is what is referred to herein as the ‘controlled onset’ for shape change behavior.

Furthermore, in various exemplary embodiments, after becoming fully hydrated, the multi-transition SME 60 becomes a hydrogel and thus undergoes a significant expansion in volume. In various exemplary embodiments, the fully-hydrated multi-transition SME 60 is a hydrogel with a diameter that is between 1.5 and 3 times the diameter of the same multi-transition SME 60 when dry. In various exemplary embodiments, the fully-hydrated multi-transition SME 60 is a hydrogel with a volume that is between 3 and 30 times the volume of the same multi-transition SME 60 when dry.

As is discussed in further detail below with regard to biomedical implantation applications, the use of PUA in the multi-transition SME 60 to impart two transition temperatures, a fixed onset for shape change transition at the second transition temperature T₂, and hydrogel qualities upon full hydration are particularly advantageous in the context of biomedical implants.

One of ordinary skill in the art could envision modifications to the above description of the multi-transition SME 60 that are considered to be within the scope of the present description. For example, the multi-transition SME 60 could comprise materials other than PGDA and/or PAA as the first polymer and the second polymer respectively, as detailed above with respect to the biocompatible SME 50. The multi transition SME 60 could also comprise a material other than PUA as the third polymer. In lieu of PUA, one can envision the third polymer being any other polymer that exhibits shape memory function, including but not limited to polyurethane, polycaprolactone, polylactic acid, polyethylene, polystyrene, polynorborene, polymethyl methacrylate, polyvinyl chloride, poly(ethylene-co-vinyl acetate), and polyimides. Polycaprolactone, polylactic acid, polyethylene glycol, and alginate in particular are believed to have significant biodegradability, and thus these and similarly biodegradable materials are held to be within the scope of the present disclosure.

### Multi-Transition SME Preparation

The multi-transition SME 60 can be prepared by first preparing the functionalized pre-polymer as described above.

The photocurable resin is prepared by combining the functionalized pre-polymer with the first reactive monomer and a second reactive monomer. In various exemplary embodiments, the first reactive monomer is acrylic acid and the second reactive monomer is 10-undecenoic acid. The photoinitiator is also incorporated into the photocurable resin.

The intrinsic shape recovery of the PGDA-PUA-PAA network 200 is triggered by internal body heat. To make the external trigger possible, additives such as magnetic and optical active particles in forms of nanoparticles and microparticles can be added into the PGDA-PUA-PAA network 200 to form a composite.

The prepared resin is processed using DLP or other 3D printing techniques such as stereolithography, liquid crystal display (LCD), extrusion-based 3D printing techniques in accordance with techniques known to one of ordinary skill in the art. For example, the DLP printing can, in various exemplary embodiments, be performed with an irradiation wavelength of 405 nm, a power density of approximately 5 mW/cm², and with a layer thickness setting of 50 μm. In various exemplary embodiments, the multi-transition SME 60 can undergo post-processing steps as known to one of ordinary skill in the art, including removal from the build platform, washing with ethanol to eliminate unreacted components, and photo-curing to complete the crosslinking process.

### Biomedical Implants

Both the biocompatible SME 50 and the multi-transition SME 60 described above are, in various exemplary embodiments, useful as materials for biomedical implants. The following is a description of how the biocompatible SME 50 uniquely enables at least one class of biomedical implants, followed by a description of how the multi-transition SME 60 uniquely enables at least another class of biomedical implants.

The biocompatible SME 50 effectively enables a biocompatible, biodegradable support material to be delivered to a treatment site inside a human body, herein called a biocompatible implant. In various exemplary embodiments, the biocompatible implant can be delivered to the treatment site as the biocompatible SME 50 in the programmed shape, which can be a shape that is optimized for ease of delivered to a treatment site in the human body, and the biocompatible SME 50 can then be triggered by heating to restore the original shape, which can be a shape that is optimized for providing physical support in treating a patient. In various exemplary embodiments, the biocompatible SME 50 can be triggered by heating from an external source known to one of ordinary skill in the art, such as a radiative heating tool, or just by the temperature of the human body. Consider an example in which a patient suffers from a weakened or collapsed blood vessel. In various exemplary embodiments, the biocompatible implant can be precisely manufactured so that the original shape of the biocompatible SME 50 serves as a scaffold matching the vessel's natural dimensions. This scaffold, while optimally shaped for its final implanted position, can be difficult to safely deliver to the treatment site, which is the exemplary patient's weakened or collapsed blood vessel. Therefore, the biocompatible SME 50 can instead be delivered in the programmed shape, which can be a compressed form optimally shaped for ease of delivery such as a rod that can fit into a catheter. Once the programmed shaped biocompatible SME 50 is delivered to the treatment site through a catheter or other minimally invasive means, the biocompatible SME 50 can then be triggered by heating above the transition temperature Tₜₐₙₛ to expand to the original form, providing temporary support while the vessel wall heals and regenerates. Once at the treatment site and transformed to the original shape, the biocompatible SME 50 stays in the original shape and maintains mechanical properties that closely match those of natural tissue due to the PGDA-PAA network 100 as described above. The elastic nature of the biocompatible SME 50 allows it to flex and move naturally with the surrounding anatomy, while its biodegradable composition enables it to gradually break down as the body's own tissue regenerates and replaces the implant. This unique combination of properties makes the biocompatible SME 50 very useful for applications that demand temporary structural support delivered through minimally invasive procedures. More detailed examples of the biocompatible SME 50 being tested as a biomedical implant are provided below in Examples 16 and 17.

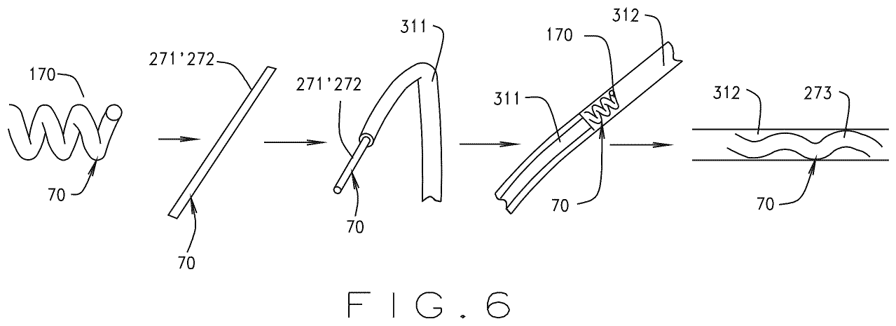

The multi-transition SME 60 enables a similar class of biomedical implants, herein called a delayed-onset implant 70. The delayed-onset implant 70 comprising a multi-transition SME 60 is in many ways similar to the biomedical implant described above, but further enables a fixed onset for shape change transition at the second transition temperature T₂, which can aid in safely delivering the delayed-onset implant 70 to the treatment site before the delayed-onset implant 70 restores the original shape. An exemplary schematic for use of the delayed-onset implant 70 is provided in FIG. 6, which again considers a patient who suffers from a weakened or collapsed blood vessel 312. The delayed-onset implant 70 is manufactured in the original shape 170, which can be a support scaffolding such as a corkscrew shape with a tubular profile. Since the original shape 170 is sufficiently large to support the blood vessel 312 but too large to fit inside of a catheter 311, the first programmed shape 271 is set as a compressed rod or other shape that easily fits within the catheter according to the programming method described above. The delayed-onset implant 70 is then delivered via the catheter 311 or other microsurgical tool to the treatment site in the blood vessel 312. The ambient temperature of the blood vessel 312 is above the second transition temperature T₂, so upon hydration, the multi-transition SME 60 begins to assume the original shape 170. The rate at which hydration of the delayed-onset implant 70 occurs determines the delay in the onset of the shape transition of the multi-transition SME 60 to the original shape 170, as described above. Finally, when fully hydrated, the delayed-onset implant 70 has retained enough water so as to have expanded and acquired the characteristics of a hydrogel 273. The hydrogel 273 is the final form of the delayed-onset implant 70. A more detailed example of the multi-transition SME being tested as a delayed-onset implant is provided below in Example 22.

In the example considered above with respect to FIG. 6, the ambient temperature of the catheter 311 is above the first transition temperature T₁, so the delayed-onset implant 70 is in the first programmed shape 271 when placed inside the catheter 311. However, in various alternative embodiments, the ambient temperature of the catheter 311 can be below the first transition temperature T₁, so the delayed-onset implant 70 can be in the second programmed shape 272 when placed inside the catheter 311 In various exemplary embodiments, the second programmed shape 272 can be the same shape as the first programmed shape 271 or a different shape. In exemplary embodiments in which the first programmed shape 271 and the second programmed shape 272 are the same shape, the multi-transition SME in either programmed shape 271/272 can be placed in the catheter 311 for delivery to the treatment site in the blood vessel 312.

The following examples are purely illustrative in nature and are not intended to be limiting of the SMEs and implants described above. The following example provide specific details of the measurements and characteristics exemplary SMEs, implants, and methods of manufacturing the same according to the present disclosure.

### SPECIFIC EXAMPLES

### Example 1: Materials and Methods

For PGDA-PAA SMEs, 4-Dimethylaminopyridine (DMAP, 99%), Glycerol (>99.5%), dodecanedioic acid (DDA, 99%), triethylamine (>99%), Dichloromethane (>99.8%), and acryloyl Chloride (>97%) were purchased from Sigma-Aldrich (St. Louis, MO, USA). ethyl acetate (99.5%), diphenyl(2,4,6-trimethylbenzoyl)phosphine oxide (TPO, >98%), and Acrylic acid (AA) (98%) were purchased from Fisher Scientific (Pittsburgh, PA, USA). 4-methoxyphenol (99%) was purchased from Acros Organics. These materials were used without further purification.

For PGDA-PUA-PAA SMEs, 4-Dimethylaminopyridine (DMAP, 99%), Glycerol (>99.5%), dodecanedioic acid (DDA, 99%), triethylamine (TEA) (>99%), Dichloromethane (>99.8%), and acryloyl Chloride (>97%) were purchased from Sigma-Aldrich (St. Louis, MO, USA). Ethyl acetate (99.5%), diphenyl(2,4,6-trimethylbenzoyl)phosphine oxide (TPO, >98%), and acrylic acid (AA, 98%) were purchased from Fisher Scientific (Pittsburgh, PA, USA). 10-Undecenoic acid (UA) (98%) was purchased from TCI. 4-methoxyphenol (99%) was purchased from Acros Organics. These materials were used without further purification.

### Example 2: PGDA Pre-Polymer Synthesis

PGD prepolymer (pre-PGD) synthesis was conducted as follows. In a three-necked flask, glycerol and dodecanedioic acid (DDA) were mixed in a 1:1 molar ratio. This mixture was then heated to 120° C. by an oil bath for 24 hours under nitrogen flow with magnetic stirring. The resulting pre-PGD had a number molecular weight (Mn) of ˜1300, a weight molecular weight (Mw) of ˜2200, and a polydispersity index of ˜1.6. To acrylate the pre-PGD to get pre-PGDA, a base solution was prepared by mixing 0.1 g of 4-methoxyphenol, 0.2 g of DMAP, 4.9 mL of triethylamine, and 200 mL of dichloromethane. This base solution was then used to dissolve 20 g of pre-PGD. After cooling the pre-PGD solution to 0° C. under nitrogen for 10 min, 3 mL of acryloyl chloride (0.18 mol/mol hydroxyl groups on the pre-PGD), which was pre-diluted in 30 mL dichloromethane, was added dropwise. The acrylation process was performed aiming to achieve an acrylation percentage of ˜18%.

After that, aluminum foil was used to seal the reaction vessel, which was then stirred at room temperature (T_{R}). After reaction for 12 hours, additional 0.1 g of 4-methoxyphenol was added. The solution was dried in a rotary evaporator by removing the dichloromethane, after which it was then dissolved in 100 mL of ethyl acetate. The supernatant was first dried in a rotary evaporator and then further dried for three days in a vacuum chamber. To separate the solubilized pre-PGDA from the triethylamine salt by-product, the mixture was centrifuged at 10,000 rpm for 10 minutes.

### Example 3: PGDA-PAA Ink Preparation and Printing Process

Pre-PGDA and AA were mixed at various pre-PGDA weight percentages of 70%, 65%, 60%, 55% and 50%. The photoinitiator diphenyl(2,4,6-trimethylbenzoyl) phosphine oxide (TPO) was added to pre-PGDA-AA resin with a concentration of 3 wt %. TPO was first dissolved in AA, and the melted pre-PGDA at 65° C. was slowly added to the AA/TPO solution while being stirred with a magnetic stir rod. After they were homogenously mixed, the resin was poured into an in-built vat of a B9Creations DLP printer (Core 550). B9Create software was used for G-code generation from the 3D models. The constructs were printed by an irradiation wavelength of 405 nm under a power density of ˜5 mW/cm² and the thickness of each layer was set to 50 μm. The printed objects were detached from the collector surface, washed with ethanol to remove the unreacted resin, and finally post-cured by 405 nm UV light for 600 s.

### Example 4: Biocompatible SME Material Characterizations

A Thermo Nicolet 380 FTIR spectrometer with DIAMOND ATR was used to collect FTIR spectra. Viscosity of resin was evaluated by a modular rotation and interface rheometer MCR302 equipped with a C60/2°. The test was performed at T_{R} with shear rates changing from 0.1 to 100 1/s. Differential scanning calorimetry (DSC) measurements were done with TA Instruments, Q-600 DSC where the temperature was decreased to −30° C. followed by ramping from −30 to 100° C. at a constant rate of 10° C./min.

Tensile tests were performed on a Mark-10ESM303 universal testing apparatus. For these tests, ASTM-D638 Type IV dog bone-shaped specimens were oriented flat against the DLP print plate, building up the sample layer by layer in the thickness direction. The initial length of the samples was 25 mm and a strain rate of 50 mm/min was applied. The cross-sectional dimensions of each sample were measured using a digital caliper for calculating the cross-sectional areas. For each set of experiments, four samples were tested both at T_{R} and above Tₜᵣₐₙₛ. to get statistical results. For the tensile tests performed above the transition temperature (Tₜᵣₐₙₛ), a heat gun was utilized to uniformly raise the temperature of each sample above its Tₜᵣₐₙₛ, which was maintained throughout the testing process. The temperature was continuously monitored using an adjacent infrared thermometer to ensure accuracy and consistency. The resulting stress-strain curves were used to calculate the mechanical properties of the printed samples. Storage modulus, loss modulus, and tangent delta were measured using Hitachi Dynamic Mechanical Analyzer (DMA7100) under the tensile mode, with printed rectangular 50 mm×10 mm×1.5 mm samples.

The shape memory behavior of the PGDA-PAA SME samples was investigated by following a typical shape memory cycling method. The sample was first stretched by 100% at a programming temperature (i.e., Tₜᵣₐₙₛ+30° C.). Then, the temperature was decreased to 25° C. After reaching the targeted programming temperature, the sample was held isothermally for 2 min. The strain of the temporary shape was measured after removing the external load. In the free recovery step, the temperature was gradually increased to the recovery temperature (i.e., Tₜᵣₐₙₛ+30° C.). The sample was held isothermally for another 1 min to observe the free recovery behavior.

For investigation of in vitro biodegradability, printed materials were submerged in 50 mL pure phosphate-buffered saline (PBS) and 50 mL PBS with 0.1 mM NaOH, respectively, at 37° C. Samples were removed at designated time points and then dried overnight at T_{R}. They were weighed after being dried to determine mass loss.

### Example 5: Biocompatible SME Cytotoxicity Testing

The biocompatibility of the printed materials was tested by co-culturing them with pluripotent mesenchymal progenitor C3H10T1/2 (10T1/2) cells in a 6-well plate for the cell counting or on coverslips in a 24-well plate for fluorescent imaging in Dulbecco's Modified Eagle Medium (DMEM) supplemented with 10% fetal bovine serum (FBS), 2 mM L-glutamine, 100 U/mL penicillin and 100 μg/mL streptomycin at 37° C. in a humidified atmosphere with 5% CO₂. Before the co-culturing assay, the printed materials were first sterilized in ethanol and then incubated in DMEM for 48 hours to diminish the effect of the materials on a medium PH value. Cell viability was tested on Day 1, 2, 3 and 4 after the co-culture, respectively. Images were captured using a Nikon microscope after each day of culturing. For cell quantification, the cells were dissociated with trypsin and then counted using a Bio-Rad TC10 automated cell counter. To take the fluorescent images, cells on coverslips were rinsed with PBS and then incubated in propidium iodide (PI) staining solution at 4° C. for 15 minutes. The samples were then protected from light, rinsed twice with PBS, and then fixed in 10% formalin at T_{R} for 10 minutes. Coverslips were then rinsed with PBS twice and mounted on glass slides with prolong gold antifade mounting medium containing DAPI. Fluorescent images were captured using a Keyence microscope and processed using ImageJ. To calculate survival rates, the number of cells positive for PI was divided by the total number of cells. Analysis on the surface of printed, degraded, and dried materials was done using scanning electronic microscopy (SEM, FEI Quanta 600F Environmental SEM).

For cell attachment assay, 13 cm disc scaffolds were printed. The discs were sterilized in 100% ethanol and then soaked in DMEM for 48 hours. 10T1/2 cells were plated with the scaffold and allowed to adhere and proliferate for 1 week. After 1 week of culture, the scaffolds were rinsed twice with PBS then fixed in 10% formalin for 1 hour, rinsed twice with PBS, and allowed to dry completely. The following day, scaffolds were prepared for SEM analysis.

### Example 6: Anastomosis of Biocompatible SME Tubes Implanted Into Mouse Aorta

The anastomosis of the 3D-printed PGDA-PAA tube implanted to the mouse aorta was conducted using a well-established aortic transplantation method in a mouse model. In preparation for the procedure, the recipient mice were anesthetized using a carefully measured blend of 1.5% (by volume) isoflurane and pure oxygen, delivered via a face mask. This was followed by a meticulous process of hair removal and disinfection in the abdominal area to minimize the risk of infection. A mid-line incision was made, stretching from the xiphoid process down to the pelvis. Special attention was given to dissect the infrarenal aorta located between the renal arteries. Any small branches off this segment were skillfully ligated using an ultra-fine 11-0 monofilament suture to prevent bleeding. Proximal and distal portions of the aorta were secured using clamps, and the intervening aortic tissue was carefully excised. The aortic lumen was then flushed with sterile saline to remove any residual blood or debris. The printed tubes were positioned in the orthotopic location, effectively replacing the removed aortic segment. Anastomosis was performed on the proximal and distal ends of the abdominal aorta, employing an end-to-end pattern using an 11-0 polyamide monofilament suture. This suturing technique ensured the secure attachment of the graft while minimizing potential damage to the vessel walls. Following the completion of the anastomosis, the clamps were gently removed to restore the aortic blood flow. The graft was closely monitored, noting the presence of visible pulses as an indication of successful blood flow restoration. The abdominal cavity was then carefully closed, ensuring that all contents were correctly repositioned to avoid any postoperative complications. The wound was closed using a 4-0 polyglycolic acid suture, which was chosen because of its strength and biocompatibility. Postoperative care included euthanizing the animals 21 days after transplantation as per the experimental design. The 3D printed tube, along with any tissues that had adhered to it, was then meticulously excised. The harvested tube material was preserved in a 4% paraformaldehyde (PFA) solution, embedded in 2% paraffin for structural stability, sectioned using a precise microtome, and subsequently analyzed using various histological stains to evaluate tissue integration and graft performance. This animal surgery procedure was conducted under the approval of the Institutional Animal Care and Use Committee of the University of Missouri (ACQA #40285), ensuring that all operations were performed under stringent ethical guidelines.

### Example 7: Biocompatible SME histology

The paraffin-embedded sections of the 3D printed tubes underwent a detailed Hematoxylin and Eosin (H & E) staining process to reveal key histological features of the adhered tissue. This staining was performed using high-quality reagents sourced from StatLab, based in Mckinney, TX, USA. Briefly, the paraffin sections were deparaffinized and rehydrated through a series of graded alcohol solutions. Once the samples were properly prepared, they were subjected to Hematoxylin staining. Hematoxylin, which colors cell nuclei a deep blue/purple, was applied to sections, allowing enough time for adequate staining. The samples were then rinsed to remove excess Hematoxylin and differentiate the staining with a bluing reagent to accentuate the color. The second component of the staining process involved the application of Eosin, which provides a pink color to the cytoplasm of the cells, creating a contrast against the Hematoxylin-stained nuclei. The sections were thoroughly rinsed again to remove any unbound Eosin. Finally, the stained sections were dehydrated through an ascending alcohol series, cleared in xylene, and mounted with a coverslip using a mounting medium. This process preserved the stained sections and prepared them for microscopic analysis. Images of these H&E-stained sections were captured using a high-resolution Nikon microscope. The microscope was meticulously calibrated to ensure accurate representation of the staining. The images were taken with a focus on areas of interest and adhered tissues to analyze the interactions between the printed tube and cells. The results of this staining and subsequent imaging provide vital information on the biocompatibility and tissue integration of the 3D printed PGDA-PAA tube. Thrombosis formation rate and material degeneration rate was calculated.

### Example 8: Immunofluorescence (IF) Staining

The immunofluorescent staining was conducted based on a protocol designed to assess the presence and location of specific cell types within the sections of the transplanted PGDA-PAA tubes. The fixed sections of the tubes underwent a deparaffinization process to remove paraffin wax used during embedding. The sections were then rehydrated using a graded series of alcohol solutions, transitioning from a high concentration down to water. This process restored the aqueous environment needed for further staining procedures. After rehydration, the sections were rinsed with Phosphate-Buffered Saline (PBS), a standard solution used to maintain the pH and osmolarity of the samples. This was followed by permeabilization with a solution of 0.5% Triton X-100 in PBS. Permeabilization is a crucial step, as it increases the permeability of the cell membrane, allowing for antibodies to penetrate the cells and bind to their targets. To prevent non-specific antibody binding, sections were blocked with a solution of 2% Bovine Serum Albumin (BSA) for 1 hour at T_{R}. BSA is a common blocking agent used to occupy potential binding sites, reducing background staining. The sections were then incubated overnight with primary antibodies against CD31 (ab222783, Abcam) or FSP-1 (S100A4) (MA5-31332, Invitrogen) at 4° C. These antibodies bind specifically to their respective antigens, allowing for detection of endothelial cells and fibroblasts, respectively. Following overnight incubation, the sections were incubated with fluorescent dye-conjugated secondary antibodies for 1 hour at T_{R}. These antibodies bind to the primary antibodies, providing a fluorescent signal that can be visualized under a microscope.

After three meticulous washes with PBS to remove any unbound secondary antibodies, the sections were mounted using an antifade reagent, which also contained DAPI (D21490, Invitrogen). DAPI is a fluorescent stain that binds strongly to DNA and is used to highlight the cell nucleus. Finally, the sections were observed under a Nikon fluorescent microscope, and images were captured. The resulting images provided a visualization of the distribution and localization of the endothelial cells and fibroblasts within the tissue adhered to the printed tube, offering insights into the biological integration of the graft.

### Example 9: In Vitro Demonstration of Transcatheter LAA Closure

The clinical applicability of the Left Atrial Appendage Occluder (LAAO) was evaluated using a computed tomography (CT) scan from a 23-year-old patient. Using the scanned data, a heart model with detailed LAA was recreated and then 3D printed. This served as the model for testing the feasibility of the uniquely designed LAAO, which was developed using Fusion 360 based on the isolated LAA from the patient's heart scan. The LAAO was 4D printed using the newly developed PGDA-PAA SME system. To visually accentuate the shape recovery of the LAAO, the 3D-printed heart model was fabricated from a transparent material. The printed LAAO, programmed into a tubular form to fit a 4 mm inner diameter tube, was then delivered to the LAA. To emulate physiological conditions and facilitate the observation of occluder recovery, the heart model was immersed in saline maintained at body temperature.

### Example 10: In Vitro Implantation of Locking Devices

The in vivo procedure necessitated the attentive preparation of a 6-month-old mouse under a rigorously controlled anesthesia administration. Using an expertly proportioned blend of 1.5% isoflurane (calculated by volume) and pure oxygen, the anesthesia was precisely delivered via a purpose-built face mask. During the procedure, the mouse's snout was carefully placed within a nose cone linked to the anesthesia system. This positioning was key in maintaining a steady state of sedation throughout the procedure, achieved by the delivery of a meticulously proportioned mixture of 1.0% to 1.5% isoflurane and 0.5 L/min 100% O₂. To confirm that the sedation was effective, a gentle toe or tail pinch was performed. The anesthesia levels were attentively adjusted, aiming to reach a target heart rate of 450±50 beats per minute (bpm), ensuring both the safety and efficacy of the surgical procedure. Preparation of the animal begins with meticulous shaving and disinfection of the ventral surface of the neck, performed via three alternating applications of 70% ethanol and betadine scrubs. The mouse was then positioned on a warming pad, and Bupivacaine was administered as a local anesthetic via subcutaneous injection at the forthcoming incision site minutes prior to making the incision. A midline incision of approximately 1.0 cm was then created on the neck's ventral surface. The locking device, which had been previously subjected to a heating process and shape-programmed to assume a flattened state at ambient T_{R}, was delicately maneuvered into place beneath the artery of the mouse, which had been surgically exposed in readiness. With an attentive and gentle approach, the heat-responsive device was positioned strategically to ensure the best results upon activation.

Following the precise positioning, a regulated amount of heat was applied, triggering the device's unique properties. It began its transformative process, gradually coiling around the aorta in a controlled and secure manner. This process was attentively monitored to guarantee the device wrapped completely and uniformly around the aorta, minimizing any potential for complications. Upon achieving its fully wrapped configuration, the device self-locked, securing itself firmly in place. Its placement was characterized by a high level of stability, firmly adhering to the contours of the aorta without causing undue pressure or damage. Post-placement, an intensive evaluation was conducted to ascertain the device's stability and fixation at the site of implantation. This critical assessment ensured the device was perfectly anchored, providing the optimal conditions for the successful continuation and conclusion of the procedure. This level of detailed scrutiny ensured that the device met all the necessary criteria for a successful and safe implantation.

### Example 11: Statistical Analysis

Experiments were repeated at least 3 times for statistical analysis. Values are expressed as the mean with standard deviation. Data values were first analyzed by comparing experimental values to control values by analyzing for Gaussian distribution using D'Agostino & Pearson and Shapiro-Wilk normality tests (alpha=0.05, p<0.05). After passing normality, parametric statistical test, unpaired t-test with Welch's correction was performed. Statistical analysis was conducted using GraphPad Prism 9 software, statistically significant differences were considered when nominal p<0.05. All p-values and the corresponding statistical tests are provided in the figure legend.

### Example 12: Description of Patient-Specific Printing Process of PGDA-PAA by DLP

Producing patient-specific, stimuli-responsive biomaterials for cardiovascular applications requires precision and thoughtful planning. While DLP presents a potential for creating high-toughness SMP at micron-scale resolution, its application to produce materials with confirmed in vitro cell studies and in vivo biocompatibility remains largely underexplored. Poly(glycerol dodecanoate) acrylate (PGDA) is a biocompatible SMP with adjustable Tₜᵣₐₙₛ, presenting a promising candidate for biomedical applications. While 4D printing of PGDA vascular grafts was reported by us, it contends with issues concerning relatively low printing resolution and low material toughness above Tₜᵣₐₙₛ. Because they were printed direct ink writing (DIW), which also suffers from a comparatively slow printing pace. In response to these limitations, herein is demonstrated digital light 4D printing of personalized biomedical implants made from PGDA-PAA SME.

It is a meticulous six-step process. First, CT scanning of a patient's heart is performed to map the intricate cardiac structures, providing a blueprint for the following steps. Armed with this precise anatomical data, a digital 3D design of an occluder is created, specifically tailored to well fit the geometry of the patient's LAA to ensure efficient occlusion. Subsequently, the designed occluder comes to life through DLP of the developed PGDA-PAA SME. After the occluder is printed, it is then subjected to shape programming, transforming it into a compacted, tubular structure that is suitable for transcatheter delivery. Once prepared, the occluder is delicately transferred via a catheter to the LAA of a printed heart model, which is a pivotal step in the MIS. The process culminates in deployment of the occluder within the LAA, accomplishing occlusion to minimize the risk of thromboembolic events. To demonstrate efficacy of this process, first the resolution was tested along with accuracy by printing free-standing, complex 3D structures: a standing man, a hand, diamond, and a locking device. They show sub-200 um features with well-matched geometries with the designed models. To demonstrate the shape programming capability, the printed locking device was tested on a tube. The devices were first elevated above Tₜᵣₐₙₛ and then programmed to a temporary flat shape. When it was placed beneath the tube and subjected to hot air, it was securely wrap around the tube. To test its in vivo capability, it was implanted into a 6-month-old mouse. It shows that the device can securely lock around the carotid artery. These results prove the device's implantability and stability within a live model, representing a significant step towards potential clinical applications.

### Example 13: PGDA-PAA Printability Assessment and Material Characterizations

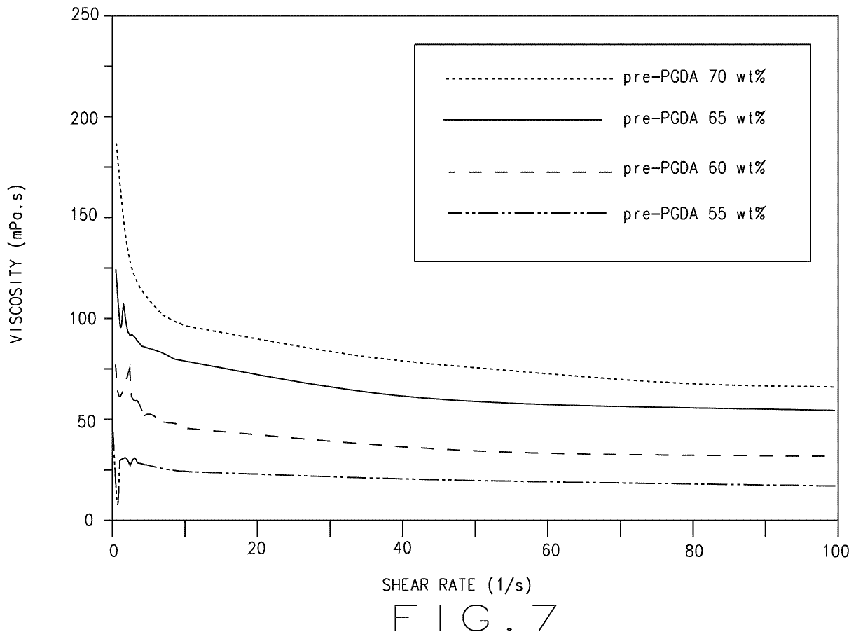

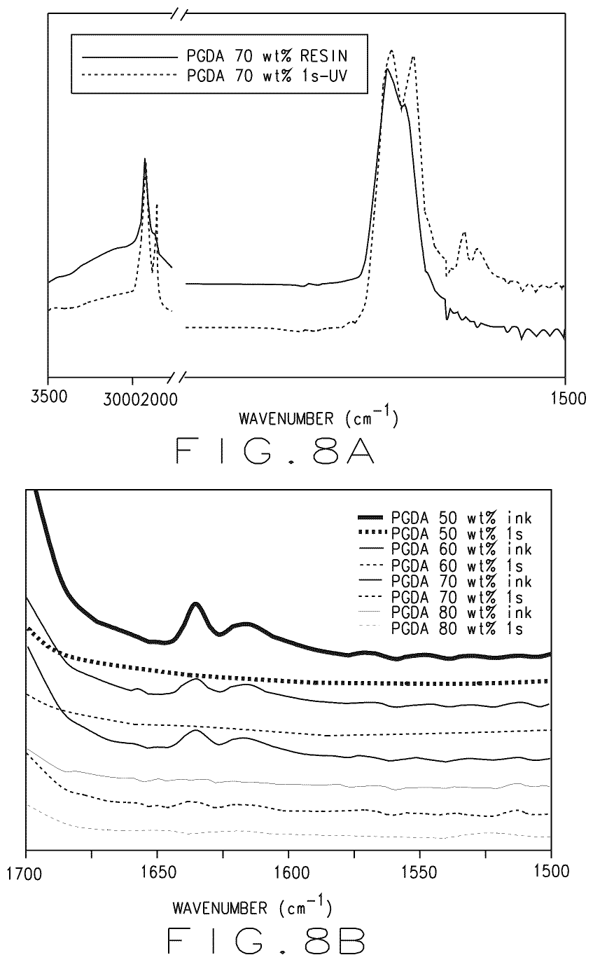

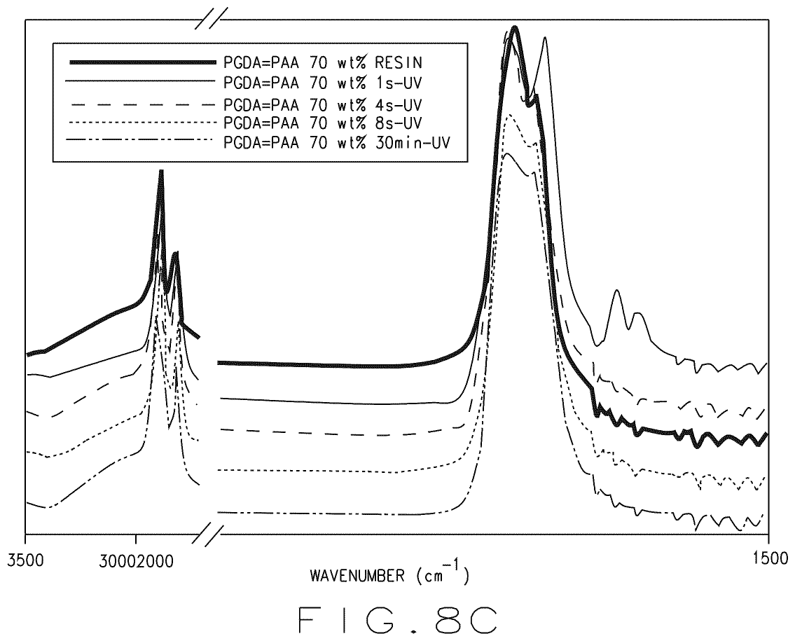

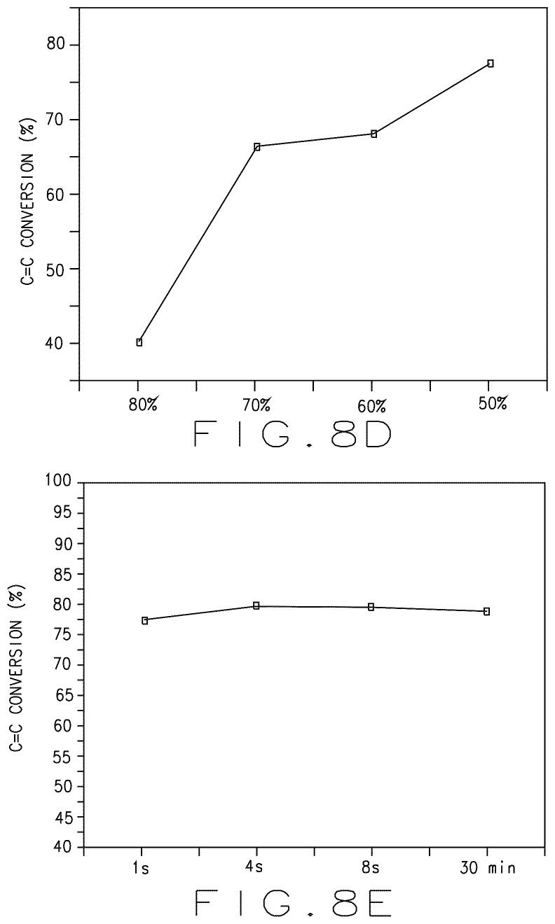

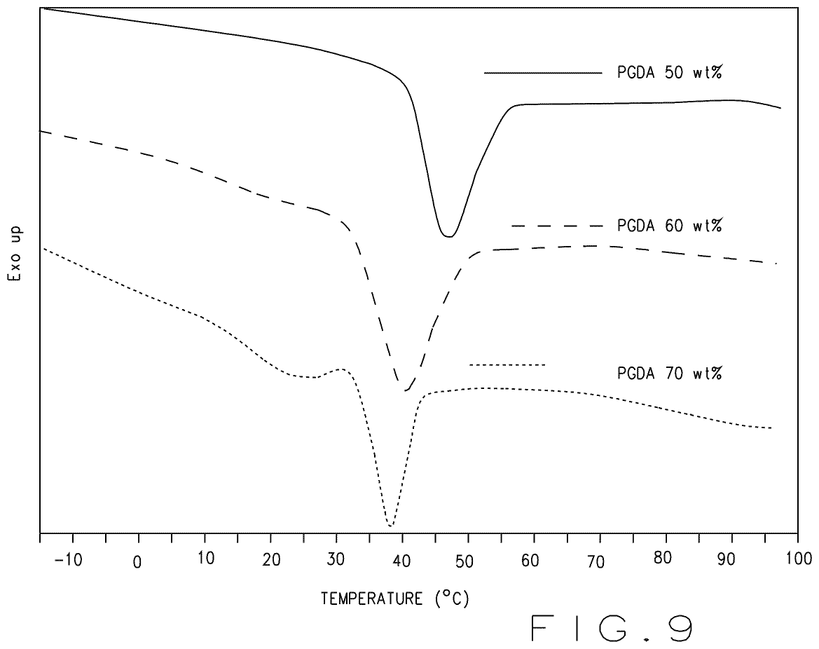

To print the 3D structures, a customized a photoactive ink consisting of pre-PGDA and AA monomer was used. The pre-PGDA was synthesized. In the formulation of the photoresin, a photoinitiator Diphenyl(2,4,6-Trimethylbenzoyl) Phosphine Oxide (TPO) was used due to its effective polymerization initiation and comparatively low toxicity, as supported by its use in biomedical applications Aware of the potential toxicity of photoinitiator derivatives, the concentration was carefully to 3 wt % and employed thorough post-processing washing steps to remove any uninitiated photoinitiator. These steps are critical for minimizing the potential long-term toxicity and ensuring the biocompatibility of the printed structures, as evidenced by the in vitro and in vivo biocompatibility results. Upon UV irradiation on the resin, TPO is activated to generate free radicals that permeate through AA and pre-PGDA. These activated precursors then undergo copolymerization to create a crosslinked PGDA-PAA network. Compatibility of the pre-PGDA/AA resin with DLP was determined by rheological and UV sensitivity characterizations. Notably, the weight ratio of pre-PGDA significantly influences the rheological behavior of the resin (FIG. 7). It shows that all resins with varied pre-PGDA weight ratios behave as a Newtonian fluid at T_{R}, independent of the testing frequency. Decrease of the pre-PGDA weight ratio from 70 to 55 wt % reduces the viscosity from 90.25 mPa·s to 23.95 mPa·s at a shear rate of 19 Hz. Nevertheless, they are still lower than the threshold value of 1.3 Pa·s for DLP. Adding AA into the precursor resin reduces the viscosity of pre-PGDA, thus improving printability. Meanwhile, presence of the resulting PAA in the PGDA-PAA network improves the elasticity and toughness, which will be discussed later. The UV-initiated crosslinking of the pre-PGDA/AA resin was monitored by ATR-FTIR (FIGS. 8A-9C), from which the conversion rates of the C═C bonds were calculated (FIGS. 8D and 9E). Intensity of the acrylate C═C peaks in AA and pre-PGDA at 1635 cm⁻¹ and 1618 cm⁻¹, respectively, was rapidly reduced within 1 s, indicating fast polymerization of the pre-PGDA/AA resins with 50 wt %-80 wt % pre-PGDA. Increase in the corresponding C═C conversion rate as the AA weight ratio increases because of the higher photo-reactivity of AA than that of pre-PGDA. The evolution of the C═C peaks as function of the UV exposure durations was also tested. As the exposure time increases, the C═C peak intensity in the pre-PGDA/AA resin with 70 wt % pre-PGDA is reduced. The resin was rapidly polymerized within the first second, with ˜80% consumption of the C═C bonds.

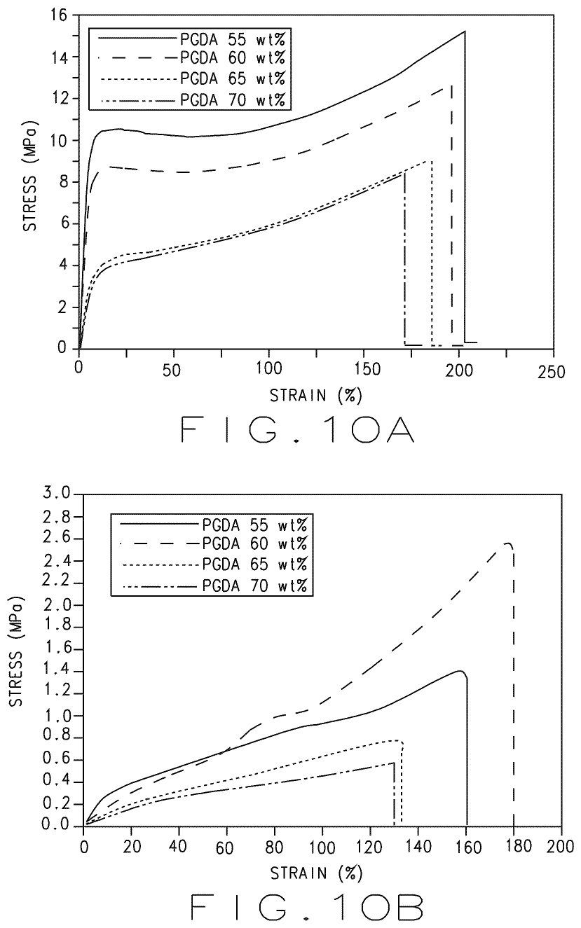

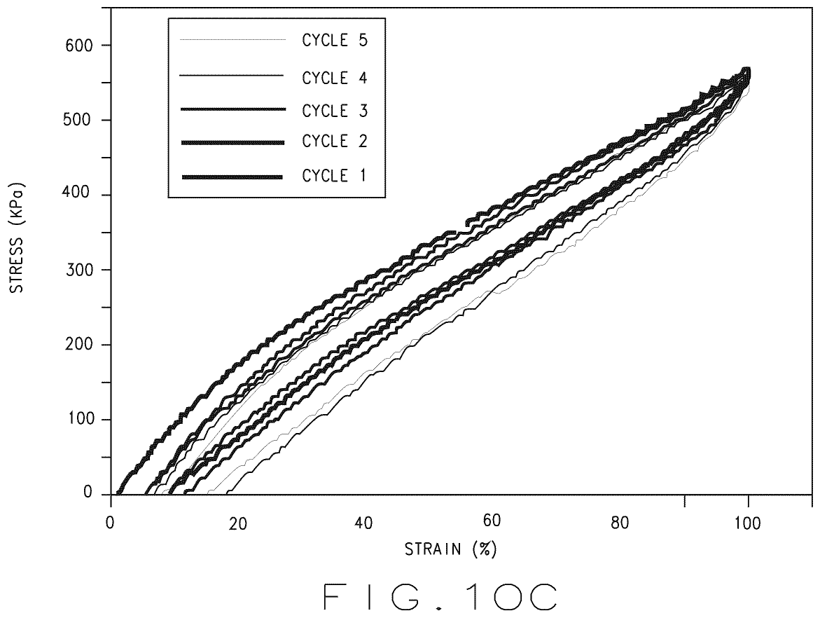

Tₜᵣₐₙₛ of PGDA-PAA was investigated using Differential Scanning calorimetry (DSC). FIG. 9 shows DSC curves of the PGDA-PAA samples with different PGDA weight ratios of 50 wt %, 60 wt %, and 70 wt %. All three curves exhibit endothermic peaks. The increase in the weight percentage of PGDA from 50 wt % to 70 wt % results in a decrease in Tₜᵣₐₙₛ from 47.2 to 39.2° C. This can be attributed to the decreased crosslinking density of PGDA-AA caused by a lowered reactivity of the resin, which results from the reduced AA content. Simultaneously, the increased PGDA content may lead to a higher degree of chain mobility, which further contributes to the reduction of Tₜᵣₐₙₛ. To investigate the effect of PGDA weight ratios on the mechanical behavior of PGDA-PAA, uniaxial tensile testing below and above the Tₜᵣₐₙₛ was performed. As shown in FIG. 10A, the tensile strength (σ_{T}) at T_{R} (below Tₜᵣₐₙₛ) decreases from 15 MPa to 7 MPa as the PGDA weight percentage increases from 55% to 70%. That can be because σ_{T} (˜5 MPa) of pure PGDA is much lower than that (˜150 MPa) of pure PAA at T_{R}. ε_{f} slightly decreases from 200% to 170% when the PGDA weight ratios increases from 55% to 70%. Above Tₜᵣₐₙₛ, similarly, both σ_{T} and ε_{f} of PGDA-PAA are reduced as the increase of the PGDA weight ratios (FIG. 10B. For instance, σ_{T} of PGDA-PAA with 55 wt % PGDA is reduced to 2.57 MPa from 15.41 MPa. But ε_{f} is well maintained 180% and 130%, slightly decreased from 200% and 170% for PGDA-PAA with PGDA weight ratios of 55 wt % and 70 wt %, respectively. This result is quite striking because normally the phase transition in SMPs significantly lowers the ductility of the material, which was also observed in pure PGDA. It is hypothesized that introduction of the linear PAA chains can augment the stretchability of the crosslinked network. Because they can be coiled and entrapped within the PGDA network. The hydrogen bonds introduced by PAA further improves the stretchability. Breaking the hydrogen bonds together with stretching the coiled linear PAA chains require much energy, thus allowing the material to withstand large deformations.

When the sample is unloaded at room temperature, some of the deformation can recover due to entropic change. However, the restoration of broken hydrogen bonds takes an extended period, leading to an observable residual strain. The residual strain can be eliminated through thermal treatment, which expedites the recovery of hydrogen bonds and enhances the mobility of the linear PAA chains. These assertions are corroborated by cyclic tensile tests above Tₜᵣₐₙₛ using PGDA-PAA samples with 70 wt % PGDA (FIG. 10C). After five continuous cycles to a strain of 100%, the sample exhibits a residual strain of 14.7%, indicating a well-maintained elasticity. This high elasticity is vital as it facilitates the shape reprogramming. A hysteresis loop is seen in the first cycle, indicating energy dissipation. The hysteresis in the second cycle is slightly lower than that of the first one. It is gradually becoming smaller as the number of cycles increases.

Correspondingly, at both below and above Tₜᵣₐₙₛ, the Young's modulus (E) and toughness (U_{T}) of the PGDA-PAA with different PGDA weight ratios were calculated from the tensile testing data. Below Tₜᵣₐₙₛ, E and U_{T} of PGDA-PAA are in the range of 40-170 MPa and 10-20 MJ/m³, respectively (FIG. 11). Increase of the PGDA weight ratios reduces both E and U_{T}. Above Tₜᵣₐₙₛ, E is significantly reduced. For instance, E of PGDA-PAA with 70 wt % PGDA decreases from 41.76 MPa to 1.02 MPa. U_{T} of PGDA-PAA with 70 wt % PGDA is also reduced from 10.18 MJ/m³ to 0.43 MJ/m³. Nevertheless, the E values above Tₜᵣₐₙₛ match well with those of the articular cartilage and cardiovascular tissue, while the toughness is 10-100 times greater than these tissues. This considerable self-driven change in the mechanical properties enables the printed 3D structures to emulate characteristics of the natural tissue while maintaining robust properties under cyclic deformations, making them apt for cardiovascular applications.

U_{T} versus E for some representative synthetic materials in clinical use can be distinctly categorized into three major groups: high-E and high-U_{T} metals and their alloys such as 316L stainless steel (SS), NiTi, Ti-4Al-6V, commercially pure titanium (cpTi), and Co—Cr, which exhibit; high-E but low-U_{T} ceramics like zirconia, alumina, and Pyrolytic carbon; and polymers including silicone, Segmented Polyurethane (SPU), Ultra-High Molecular Weight Polyethylene (UHMWPE), Polytetrafluoroethylene (PTFE), Polyamide 6-6 (nylon 6-6), Polyethylene Terephthalate (PET), Polyether Ether Ketone (PEEK), Polylactic Acid (PLA), and Poly(Methyl Methacrylate) (PMMA) which demonstrate a wide range of E and U_{T} values. This categorization underpins the rationale behind the material choice for specific biomedical applications, such as the preference for metals in joint replacements due to their high-E and high-U_{T}. An interesting trend is that clinically successful materials generally possess high toughness. In fact, U_{T} (10-100 MJ/m³) of most synthetic materials surpasses those (0.01-10 MJ/m³) of most living tissue. Notable exceptions include PMMA, PLLA, and ceramics, each with unique attributes that justify their use in specific circumstances. For instance, PMMA is often used as bone cement, while PLLA, despite its lower toughness, is valued for its biocompatibility. Additionally, PCL is an FDA-approved high-toughness polymer that shows no shape memory behavior at body temperature. Nevertheless, there remains a need for biomaterials with high U_{T}, suitable E and additional functionalities such as shape programming response for MIS involving soft tissue. Bridging this gap is a challenge due to the intrinsic trade-offs between E and U_{T}. The PGDA-PAA SME holds a great potential to address this challenge.

### Example 14: Thermomechanical Performance of PGDA-PAA

We also investigated the thermomechanical properties of PGDA-PAA. To program the sample to a temporary shape by strain, it first is heated above Tₜᵣₐₙₛ. PGDA-PAA, cross-linked as described above, exhibits rubber-like elasticity above Tₜᵣₐₙₛ and can be easily deformed with an external force to form a new secondary shape. Then this programmed shape is cooled below Tₜᵣₐₙₛ. When the sample is cooled, the crystalline domains, dominated by PGDA, percolate to fix the strained shape. This temporary shape (though possibly subjected to prolonged warpage) is maintained below Tₜᵣₐₙₛ. When heated above Tₜᵣₐₙₛ again, the PGDA crystalline domains melt to form an amorphous, homogeneous phase with high mobility, allowing the fixed shape to recover to the original shape. The role of PGDA in PGDA-PAA is to impart the shape memory effect into the 3D printed structures to realize 4D printing. The PAA chains improve the ductility of PGDA-PAA while maintaining the high tensile strength at both below and above Tₜᵣₐₙₛ. As presented in FIG. 2, a programmed 3D structure recovers to its as-printed shape when heated above Tₜᵣₐₙₛ within 25 seconds.

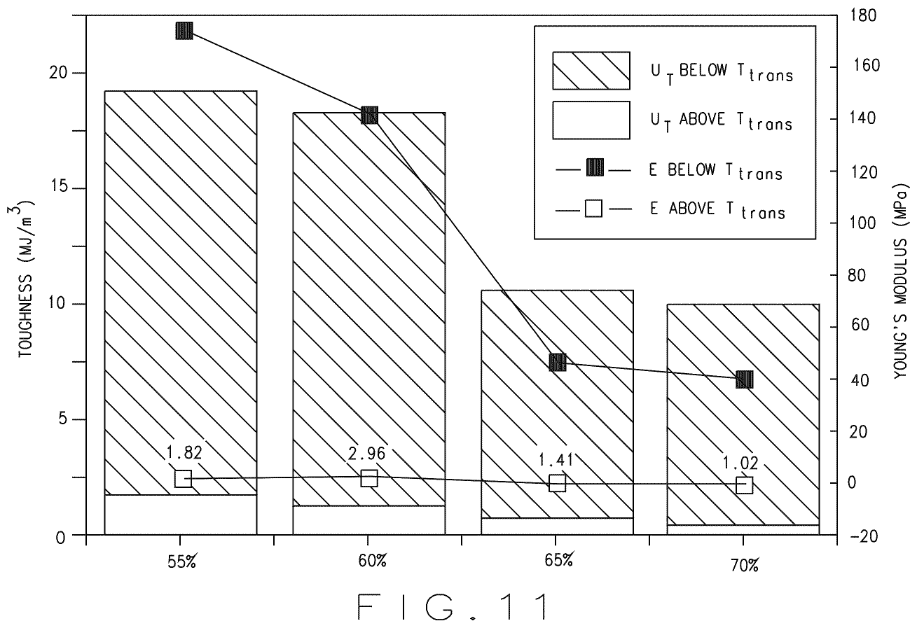

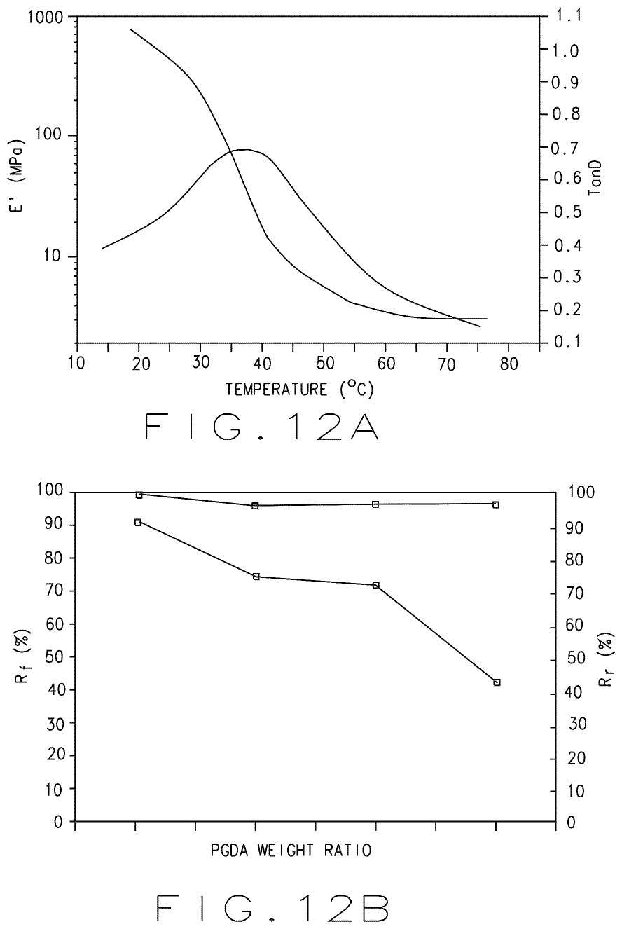

FIG. 12A presents DMA results for the PGDA-PAA SMEs with varying weight ratios of PGDA. It displays the temperature-dependent storage modulus, which corresponds to a sample's elastic response. It shows a clear drop in the storage modulus when the temperature is above Tₜᵣₐₙₛ. Additionally, FIG. 12A plots the temperature-dependent TanD, which is defined as the ratio of loss modulus (corresponding to energy dissipation) to the storage modulus. The curve shows transition temperature at its peak for a polymer, such as the glass transition temperature for an amorphous one and the melting/crystallization temperature for a semi-crystalline one. The peak in the curve for the PGDA-PAA SME with 70 wt % PGDA appears at transition temperature of 39.38° C., which closely agrees with the value obtained from the DSC test (FIG. 9).

We further investigated the effect of PGDA weight ratios on the shape memory (SM) behaviors of the PGDA-PAA samples. To quantify the SM behaviors, the shape fixity ratio (R_{f}) as R_{f}=εᵤ/εₘ×100% and the shape recovery ratio (Rᵣ) as Rᵣ=(εᵤ−εᵣ)/εᵤ×100% were calculated, where εₘ is the maximum strain before unloading, εᵤ is the strain immediately after unloading, and εᵣ is the instantaneous strain after recovery. In the evaluation, they were first stretched by a 100% strain at 45° C. After being cooled to T_{R}, their shapes were fixed. Reheating the samples above 45° C., the shapes are supposed to recover back to the original ones. As shown in FIG. 12B, with the decrease of the PGDA weight ratio from 70 wt % to 55 wt %, R_{f} is significantly decreased from 92% to 43%. It can be expected because R_{f} of a semi-crystalline SMP is highly related to the crystalline regions. An increase in the PGDA ratio results in a higher amount of crystalline regions in the SME system, leading to improved R_{f}. In contrast, all the PGDA-PAA samples with different PGDA weight ratios exhibited R_{f} of >95%, and the increased PGDA ratio only slightly decreases R_{f} (FIG. 3B). After balancing the mechanical and the thermomechanical properties, it was decided to use the PGDA-PAA SME with 70 wt % PGDA as the material for the following in vitro and in vivo studies.

### Example 15: Cytocompatibility of PGDA-PAA

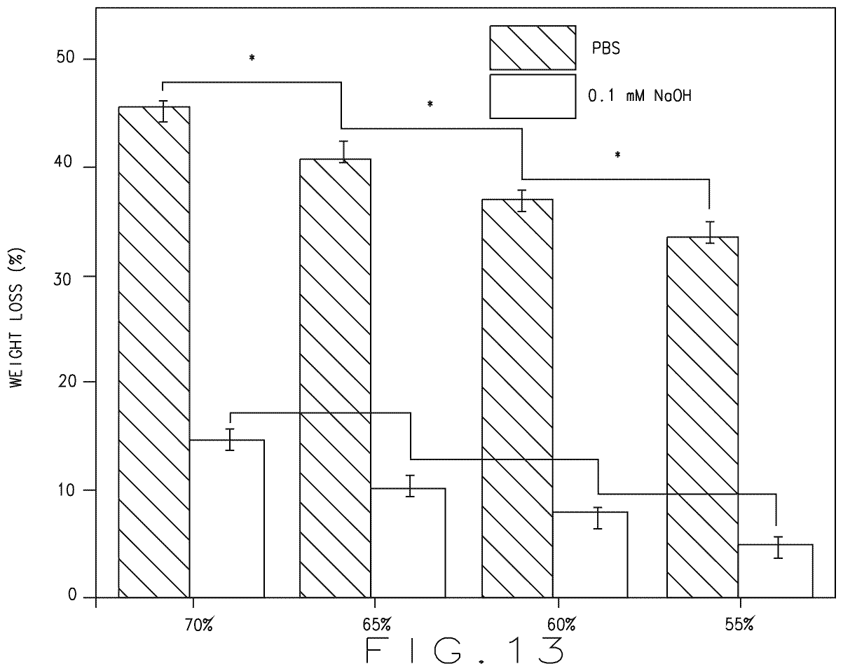

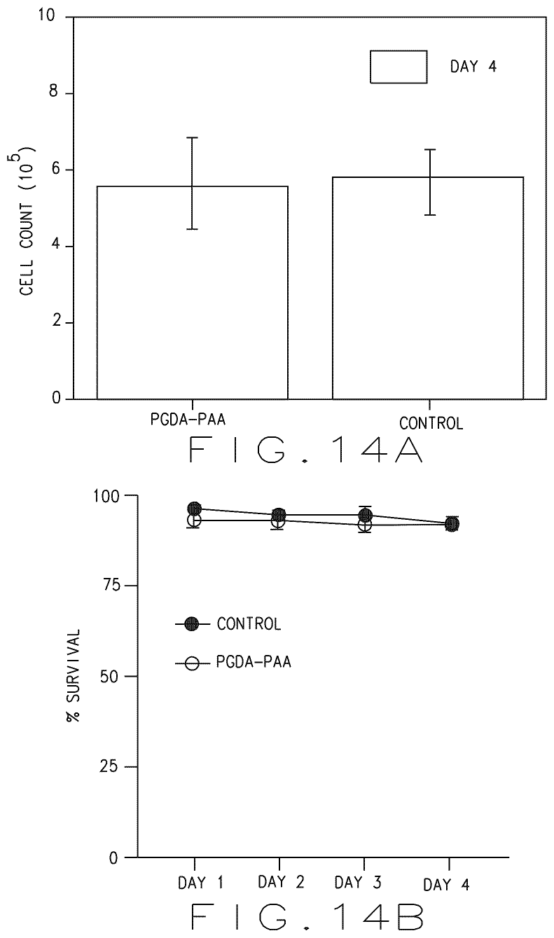

Upon implantation, PGDA-PAA is expected to provide temporary support for cell growth until it is replaced by regenerated tissue. Therefore, biodegradability and biocompatibility are crucial for this purpose. As shown in FIG. 13, in six-month degradation experiments, the printed PGDA-PAA with 70 wt % PGDA exhibited 45% weight loss and 15% weight loss in 0.1 mM NaOH solution and PBS, respectively. As the PGDA weight ratio decreases, the degradation rate decreases. It can be expected because PGDA is a polyester, which is more prone to be degraded than PAA is. SEM images of the PGDA-PAA samples show severe surfaces erosion after 6-month degradation in both 0.1 mM NaOH and PBS solutions). The cytocompatibility of PGDA-PAA was assessed using 10T1/2 cells co-cultured in vitro. Stereo microscope images taken over the four-day culturing period show significant cell proliferation. FIG. 14A indicates that the number of the grown cells co-cultured with PGDA-PAA is close to the control with a 2.5% difference, suggesting biocompatibility of PGDA-PAA. An unpaired t-test shows statistical similarity between the cell count of co-cultures with printed materials and the control with a P value equal to 0.8761. Propidium Iodide (PI) and DAPI staining showed no significant difference in the cell survival among the groups. The cell survival rates were quantified to be >90% after four days of cell culture (FIG. 14B), indicating that PGDA-PAA is cytocompatible.

We also investigated how the PGDA-PAA surface topography affects cell morphology and the way cells adhere, grow, and differentiate. After an incubation period of 7 days, the used 10T1/2 cells appeared to well adhere to the PDGA-PAA surface. In addition, they spread widely and formed lamellipodia at the leading edge. The cells also developed short filopodia at their apical poles, spreading from the lamellipodia, which is consistent with the properties as fibroblast-like cells. The lamellipodia is a dense network of cross-linked actin filaments that drive cellular distribution and motility. The filopodia are exploratory extensions formed via parallel bundles of actin filaments from the plasma membrane. The thin actin protrusions can probe the extracellular environment to guide cell migration towards specific sites of interest. As filopodia is involved in cellular processes of wound healing, extracellular matrix adhesion, chemoattractant guidance, and neuronal growth-cone pathfinding, formation of the filopodia, which senses cell surrounding and acts as sites for signal transduction, is important for directing cell migration on the PDGA-PAA surface. These results prove that the surface topography directly affects the extension and adhesion of the filopodia and lamellipodia.

### Example 16: In Vivo Study of a Printed PGDA-PAA Vascular Graft

To test if a printed PGDA-PAA tube can serve as a biocompatible scaffold for vascular grafting, the tube was implanted onto mouse aorta through an end-to-end anastomosis. An implanted tube featuring a height of 15 mm, an outer diameter of 2 mm, and a wall thickness of 150 μm was designed to facilitate the grafting procedure. After 21 days, tissues began to adhere to both the inner and outer surfaces of the implanted tube. Gross images and H&E staining of the tubes revealed thrombosis formation in the lumens of a few tubes, and material degradation over time. Occurrence of the thrombosis and post-implantation material degradation are important considerations when evaluating the value of a vascular graft. Thrombosis is a significant clinical concern in vascular grafts, as it can obstruct blood flow and compromise graft function. It is noteworthy that thrombosis is a common complication following surgical procedures, including aorta transplantation. This is true even for FDA-approved artificial aortas used in aneurysm repair surgeries. In this study, the formation of thrombi was observed after 21 days post-implantation, as indicated in the H&E staining image. Thrombi appeared in the lumen of the graft, potentially as a response to the foreign material or due to alterations in blood flow caused by the graft. The observed thrombosis rate of ˜25% is comparable to, if not lower than the rates reported for other materials used in similar contexts, suggesting that the 4D printed vascular grafts did not excessively promote the thrombus formation. Nevertheless, Continuous monitoring of thrombosis rates is essential for understanding the long-term performance and safety of the vascular graft. Material degradation is another critical factor when assessing biocompatible scaffolds for tissue engineering. Degradation allows for the scaffold to be gradually replaced by natural tissues, leading to a more natural, functional graft. H&E staining revealed signs of material degradation, suggesting that the PGDA-PAA tube is not only biocompatible but also biodegradable. This is advantageous as it means that the graft can potentially be replaced by the patient's own tissue over time, improving an overall integrity and function of the repaired vessel. Both thrombosis and material degradation highlight the dynamic interaction between the implanted graft and the surrounding biological environment. Understanding these processes in depth will be crucial for further development and improvement of the PGDA-PAA vascular grafts.

On the outer surface of the tube, cell migration was evident, as was growth of the adventitia. Immunostaining revealed CD31-positive endothelial cells lining the inner surface of the tube, indicative of a biocompatible environment conducive to endothelial cell growth and endothelium formation. This is crucial for maintaining vessel integrity, preventing coagulation, and controlling blood flow. The outer surface of the tube was populated with FSP-1-positive fibroblasts, suggesting the development of a tentative adventitial layer. The combination of CD31-expressing endothelial cells and FSP-1-positive fibroblasts signifies the beginning stages of medial and adventitial layer formation around the graft. Given the complexity and functionality of native arteries and veins, these results highlight the promising potential of the synthetic PGDA-PAA materials for arterial grafting applications. In this study, a mouse model was employed for the biocompatibility testing of the bioresorbable shape memory elastomers. While this model offers advantages such as ease of handling, cost-effectiveness, and established experimental protocols, there are some limitations. There exist significant anatomical and physiological differences between mice and humans, including smaller vessel sizes, distinct blood pressure levels, and different arterial wall properties, which may potentially affect the outcomes of the implantation procedure and the relevance to human conditions. Furthermore, the progression of diseases and healing processes in mice may not accurately mimic the human pathophysiology. Transitioning from a mouse model to human involves more than just scaling up device sizes, but requires careful consideration of biomechanical properties, blood flow dynamics, and tissue response.

### Example 17: In Vivo Study of a Printed PGDA-PAA Vascular Graft

A CT scan from a 23-year-old female patient, with a medical history inclusive of hypertension, hyperlipidemia, diabetes, and smoking, was utilized for the dual purpose of designing the left atrial appendage (LAA) occluder (LAAO) and 3D printing a model of the heart model. This personalized LAAO design was then printed with PGDA-PAA. The patient's heart, including the LAA, was included in the heart's 3D model. The model was printed by a transparent elastomer that emulates the mechanical properties of heart tissue, thereby enhancing the realism of in vitro trans-catheterization feasibility studies. The in vitro test of transcatheter LAA closure procedure was executed using a silicone tube to mimic the role of a catheter, delivering the LAAO to the LAA of the printed heart immersed in saline maintained at physiological temperature. Before delivery, the LAAO was programmed to a compact, temporary shape above Tₜᵣₐₙₛ and then was fixed under Tₜᵣₐₙₛ. The LAAO can be smoothly extruded from the catheter, underscoring the potential for minimally invasive implantation. Upon being released near the LAA orifice, the shape of the LAAO was recovered to the originally printed shape. For a more tangible representation of LAA occlusion effectiveness, red coloring was introduced into the saline as an analog for colored saline that might diffuse into the LAA post-LAAO deployment. It shows that the LAAO can effectively enclose the LAA, indicating a potential clinic application in future. However, it is imperative to clarify that the demonstration is just an early-stage proof-of-concept. The development of a ready-to-use implant entails a series of intricate and rigorous studies that have yet to be performed.

### Example 18: Synthesis of PGDA Oligomer (or Pre-PGDA) for PGDA-PUA-PAA

PGD oligomers, the PGDA precursor, were first synthesized. To do that, a three-necked flask was used to mix glycerol and DDA in a 1:1 molar ratio. In an oil bath with nitrogen flow and magnetic stirring, the mixture was heated to 120° C. for 12 and 24 hours to get PGD-12 and PGD-24 oligomers with molecular weights of 1026/1698 g/mol and 1026/1698 (Mₙ/M_{w}). The PGD-12 and PGD-24 oligomers were then acrylated by acryloyl chloride to synthesize PGDA 12H (High acrylation), PGDA-12L (Low acrylation), and PGDA-24 oligomers, or named pre-PGDA. Briefly, 30 g of PGD oligomer was dissolved in 300 ml of dichloromethane in a 500 ml round bottom flux. Then 0.15 g of 4-methoxyphenol, 0.3 g of DMAP, and TEA (with 1:1 molar ratio of acryloyl chloride) were added to solution. After cooling the solution to 0° C. under nitrogen flow for 10 min, acryloyl chloride was dropwise diluted in dichloromethane (10× of acryloyl chloride volume). After that, aluminum foil was used to seal the reaction vessel, which was then stirred at room temperature. After the reaction for 12 hours, additional 0.15 g of 4-methoxyphenol was added to the solution. The solution was dried in a rotary evaporator by removing the dichloromethane, and then dissolved in 150 mL of ethyl acetate. To separate the solubilized PGDA oligomer from the triethylamine salt by-product, the mixture was centrifuged at 10,000 rpm for 10 minutes. To get PGDA oligomer, the supernatant was first dried in a rotary evaporator and then dried for three days in vacuum.

### Example 19: 3D Printing of PGDA-PUA-PAA

PGDA-12L oligomer, UA, and AA were mixed at various ratios of 20:50:30, 35:35:30, and 20:50:30 (percentage). Photoinitiator of diphenyl(2,4,6-trimethylbenzoyl)phosphine oxide (TPO) was added to PGDA-UA-AA resins at a concentration of 3 wt %. Specifically, TPO was first dissolved in AA, then the melted UA and melted PGDA oligomer at 65° C. were added to the solution while being stirred by a magnetic stir rod at 65° C. After all components were homogenously mixed, prepared resins were poured into the in-built vat of the B9Creations DLP printer (Elite Micro) with an irradiation wavelength of 385 nm and exposure of 50 mJ/cm². The layer thickness was set to 20 μm. The printed objects were detached from the surface, washed with isopropanol to remove the unreacted resin, and finally post-cured by 405 nm UV light for 10 min.

Synthesis of Poly(glycerol dodecanoate acrylate) (PGDA) oligomers with various molecular weights and acrylation degrees detailed synthesis conditions and resulting materials are summarized in Table 1. As the reaction time increases from 12 hours to 24 hours, the molecular weights (Mₙ/M_{w}) of PGD oligomers, the PGDA precursor, increase. The acrylation degrees were calculated based on the characteristic peaks in ¹H NMR spectra. To make the SMP 3D printable by DLP, a photocurable resin was formulated, consisting of PGDA oligomers, 10-Undecenoic acid (UA), and acrylic acid (AA). Choice of PGDA is based on its ability to establish a phase transition above body temperature. To circumvent the challenge of low photoactivity and low elasticity of PGDA, PAA was introduced in the PGDA-PAA network previously described. But it was found that if water was uptaken by PAA, PGDA-PAA showed significantly diminished elasticity due to reduced polymer-polymer interactions in the hydrated state. To enhance the elasticity, herein, UA is incorporated in the ink formulation. Strikingly, this addition brings in two benefits. Firstly, another phase transition temperature (Tₜᵣₐₙₛ₁) is introduced by PUA besides the one introduced by PGDA (Tₜᵣₐₙₛ₂) as shown in the photocrosslinked network. Differential Scanning calorimetry (DSC) curves of the PGD oligomer, PGDA oligomers and resulting PGDA-PUA-PAA were acquired. They highlight the impact of molecular weight and acrylation degree on the transition temperatures (Tₜᵣₐₙₛ₁ and Tₜᵣₐₙₛ₂) of PGDA-PUA-PAA. A lower acrylation degree (21%) in PGDA-12L oligomer yield a lower crosslink density, resulting in Tₜᵣₐₙₛ₁ of ˜20° C. and Tₜᵣₐₙₛ₂ of 40° C., attributed to the semicrystalline PUA and PGDA segments, respectively. In contrast, higher acrylation degrees in PGDA-12H and PGDA-24 oligomers (41% and 30%) increase the crosslinking densities, causing diminished both transition temperatures in PGDA-PUA-PAA. Thus, to preserve the second phase transition in PGDA-PUA-PAA, in the rest of the study, the PGDA-12L oligomer was chosen as the ink precursor. Secondly, PUA is hydrophobic and self-assembles to a phase separated from the one formed by the hydrophilic PAA by hydration. This phenomenon is discussed in later sections. Note: Tₘ is melting temperature of the synthesized PGDA oligomers.

TABLE 1
PGD and PGDA oligomer synthesis conditions and properties

| | PGD oligomer Synthesis | | | |
|---|---|---|---|---|
| | Conditions | | Properties | |
| | Molar ratio of | Reaction duration, | Mₙ/M_{w} | Tm |
| | DDA:Glycerol | Temperature | (g/mol) | (° C.) |
| PGD-12 | 1:1 | 12 h, 120° C. | 1026/1698 | 50 |
| PGD-24 | 1:1 | 24 h, 120° C. | 2085/5556 | 52 |

| | PGDA Oligomer Synthesis | | | |
|---|---|---|---|---|
| | Conditions | | | |
| | Molar ratio of | | | |
| | Hydroxyl | | Properties | |
| | group:acryloyl | Reaction duration, | Acrylation | Tₘ |
| | chloride | Temperature | degree (%) | (° C.) |
| PGDA-12L | 1:0.45 | 12 h, 25° C. | 21 | 38 |
| PGDA-12H | 1:0.9 | 12 h, 25° C. | 41 | 50 |
| PGDA-24 | 1:0.48 | 12 h, 25° C. | 30 | 52 |

To elucidate how the acrylation and molecular weights modulate the thermal transitions, the printability of the precursor characteristics was investigated by Attenuated Total Reflectance Fourier Transform Infrared Spectroscopy (ATR-FTIR)). The FTIR spectra of the formulated photoresin with various ink composition ratios reveal that the acrylate C═C bonds undergo rapid conversion within the first second of UV exposure. This rapid polymerization supports high-resolution printing. Moreover, the C═C bond conversion rate increases with higher PGDA oligomer weight ratios, attributed to the higher photo-reactivity of PGDA oligomer compared to UA, enhancing the resin's reactivity for sustained performance under prolonged UV exposure. The dual transition temperatures of the dry sample and hydrated sample (hydrogel) are exhibited in the DSC curves (FIG. 5). For the dry sample with a PGDA:PUA:PAA weight ratio of 50:20:30, before hydration, the curve exhibits two peaks at ˜17° C. (Tₜᵣₐₙₛ₁) and ˜40° C. (Tₜᵣₐₙₛ₂). After hydration, these two peaks downshift to ˜14° C. and ˜34° C., respectively. This behavior with lowered T_{g} was observed in polymers such as hydrophilic polyurethane (PU), originating from the plasticizing effect of water. Ramdas et al. confirmed that the solvent-driven shape changing was caused by the plasticizing effect of the absorbed solvent molecules. However, the solvent-driven SMPs are limited by prolonged recovery times and low recovery ratios due to slow solvent diffusion. To address these limitations, hydrophilic PAA was copolymerized with semicrystalline PGDA and PUA to make rapid hydrothermal activation driven by the physiological temperature first followed by the fluid activation. This combined approach is more controllable than previous known single-activation methods. Moreover, this proposed SMP has a shape recovery onset property, which is discussed below.

### Example 20: Materials Characterization for PGDA-PUA-PAA

¹H NMR Test: To calculate the acrylation degree, proton nuclear magnetic resonance (1H NMR) was used. The data was recorded at room temperature using a Bruker Avance III 500 MHz spectrometer with tetramethylsilane (TMS) and chloroform-d serving as the internal reference and solvent at peak of 7.27 ppm, respectively. Chemical shifts were reported in ppm.

Gel Permeation Chromatography (GPC). The molecular weights of PGD-12, and PGD-24 oligomers were determined using gel permeation chromatography (GPC) on an Agilent 1200 Series high-performance liquid chromatography (HPLC) system (Agilent Technologies, Palo Alto, USA) equipped with an IR detector. The analysis was conducted with an Agilent PLgel 5 μm MIXED-D column (300×7.5 mm), maintained at 35° C. Tetrahydrofuran was used as the mobile phase at a flow rate of 1 mL min⁻¹. Molecular weight calculations were based on a calibration curve constructed using polystyrene standards with molecular weights ranging from 266 to 66,000 g mol⁻¹.

ATR-FTIR Spectroscopy. A Thermo Nicolet 380 FTIR spectrometer with DIAMOND ATR was used to collect the FTIR spectra.

FTIR Chemical Imaging. A PGDA-PUA-PAA (35:35:30) film was drop cast onto a silicon substrate followed by UV exposure. Chemical imaging of the samples was performed using a Bruker Invenio FTIR spectrometer equipped with an FTIR microscopy Hyperion Il system by a reflectance mode using a 36× reflectance IR objective. These experiments were conducted on dry samples followed by being immersed in DI water for durations of 1, 5, 10, and 20 minutes. At each of these time points, 100 FTIR spectra were collected from 100 different points with a spatial resolution of 40 μm on the samples. The spectra were collected using Bruker OPUS software, which also captured the optical images of each sample. The FTIR spectra were extracted and processed in MATLAB to produce chemical maps. To isolate the chemical signals related to hydrogen bonding from hydration-induced phase separation, the difference spectroscopy (DS) technique was applied. This approach removes spectrum signals from the dry polymer and focuses on the changes induced by water. The difference spectra were calculated by subtracting the spectra of the dry samples from the spectra of the wet samples at each time point by the equation of A_{d}(n)=Aₛ(n)−k·Aᵣ(n), where A_{d}(n) is the absorbance of the difference spectrum at frequency n, Aₛ(n) is the absorbance of the spectra from wet specimen, and Aᵣ(n) is the absorbance of the reference spectrum from the dry specimen. The parameter k compensates for any thickness differences between the samples. The k-value was calculated by analyzing peaks in regions unaffected by water (outside of 3,800-3,400 cm⁻¹). Once the k-value was determined, the DS procedure was applied to get signal difference in the frequency ranges of interest: 3,800-3,400 cm⁻¹ for hydroxyl group. These generated chemical maps revealed how H-bonds were formed and how phase separation was induced by water over time by analyzing the spatiotemporal distribution of H-bonds, which offered a clear view of the dynamic phase separation processes in the PGDA-PUA-PAA material.

Differential Scanning calorimetry (DSC). DSC measurements were taken with TA Instruments, Q-600 DSC. First, for dry samples, the temperature was decreased to −30° C. and then increased to 100° C. at a rate of 10° C. min⁻¹. For the swelled samples, the temperature was decreased to near 0° C. and then ramped from 0° C. to 100° C. at a rate of 10° C. min⁻¹. Cooling to near 0° C. was implemented to avoid the melting endotherm of water, thus allowing the detection of the sample's first transition temperature without overlap from water-related thermal events.

Scanning Electron Microscopy (SEM). SEM imaging on the samples was done by FEI Quanta 600F Environmental SEM. Samples were prepared as follows: PGDA-PUA-PAA with four different ratios (70:0:30, 50:20:0, 35:35:30, and 20:50:30) were immersed in deionized water for 24 hours. To enable the cross-section imaging of each sample, the swelled samples were cleaved by putting in liquid nitrogen and then freeze dried.

Mechanical Characterizations. Tensile tests were performed on a Mark-10 F305-EM universal testing apparatus. Printed specimens with an ASTM-D638 Type IV dog bone shape were used for tensile testing. The initial length of the samples was 25 mm and a strain rate of 50 mm/min was applied. The cross-sectional dimensions of each sample were measured using a digital caliper for calculating the cross-sectional areas. For each set of experiments, four samples were tested. The resulting stress-strain curves were used to calculate the mechanical properties. Storage modulus was measured using Hitachi Dynamic Mechanical Analyzer (DMA7100) with 3D printed rectangular 50 mm×10 mm×1.5 mm samples.

Swelling Test. For swelling test, PGDA-PUA-PAA samples with the weight ratio of 50:20:30 were cut into a rectangular shape (1.5 cm*1 cm*0.15 cm). They were immersed in PBS solution and DI water in 37° C., and room temperature (RT) to investigate effects of temperature and solution on swelling induced volume expansion. The volume expansion of the printed coil was assessed by measuring the diameter increase from the photographed images before and after swelling. The final volume of the swollen coil was then determined using the properties function in Fusion 360.

Cytotoxicity of Materials. The biocompatibility was tested by co-culturing with pluripotent mesenchymal progenitor C3H10T1/2 (10T1/2) cells. Cells were cultured and placed in a 6-well plate for the cell counting and on coverslips, a 24-well plate for fluorescent imaging in Dulbecco's Modified Eagle Medium (DMEM) supplemented with 10% fetal bovine serum (FBS), 2 mM L-glutamine, 100 U/mL penicillin and 100 μg/mL streptomycin at 37° C. in a humidified atmosphere with 5% CO₂. Before the co-culturing assay, the printed material was first sterilized in ethanol and then incubated in DMEM for 48 hours. Cell viability was detected on Days 1, 2, 3 and 4. Images of cells on each group of samples were captured using a Nikon microscope at each day of the culturing for four days. To do cell quantification, the cells were dissociated with trypsin and then counted using a Bio-Rad TC10 Automated Cell Counter. For fluorescence images, cells on coverslips were rinsed with PBS and then incubated in propidium iodide (PI) staining solution for 15 minutes at 4° C. The samples were then protected from light, rinsed twice with PBS, and then fixed in 10% formalin for 10 minutes. Coverslips were then rinsed with PBS twice and mounted on glass slides with Prolong Gold antifade mounting medium containing DAPI. Fluorescence images were captured using a Keyence microscope and processed using ImageJ. To calculate the percent survival, the number of cells positive for PI was divided by the total number of cells.

### Example 21: PGDA-PUA-PAA SME Synthesis and Characterization

Using a formulated ink as described above, a PGDA-PUA-PAA micro coil with a diameter of 600 μm and volume of 3.737 mm³ was printed. To probe the shape memory property, the printed micro coil was programmed into two temporary shapes. To do that, it was first heated above Tₜᵣₐₙₛ₂ and then mechanically deformed into an arch shape followed by cooling it to temperature between Tₜᵣₐₙₛ₁ and Tₜᵣₐₙₛ₂. In this same temperature, the shape was deformed to a linear shape followed by cooling the temperature to room temperature (RT) to fix this shape. Both shape fixity ratios reached almost 100%. The two-shape programming is essential to demonstrate the material's ability to fix and maintain the shapes for applications where the coils or stents adapt to varying storage, deployment conditions and physiological temperatures. After the shape programming, the sample with the linear shape was transferred to a chamber with a controlled temperature of 37° C. At a dry state, the linear shape was first recovered to the programmed arc shape within 4 mins. Then phosphate-buffered saline (PBS) was dropped to immerse the sample, this intermediate shape was maintained for ˜6 mins, which is called a shape recovery onset duration. Subsequently, the sample uptook water and swelled, which reduces Tₜᵣₐₙₛ₂ to 34° C. This reduced Tₜᵣₐₙₛ₂ below 37° C. triggers the second shape recovery to the originally printed coil shape. As the swelling continued, the coil was finally transformed to a hydrogel at 37° C. with a diameter expansion of 2.8 times and volume expansion of 22 times due to abundant carboxylic and hydroxyl groups in the PAA segments.

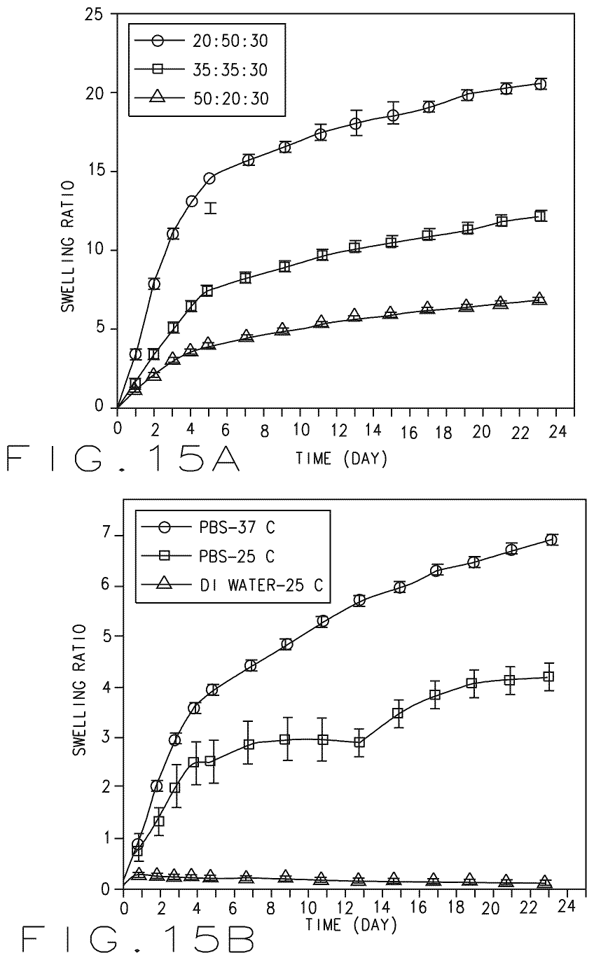

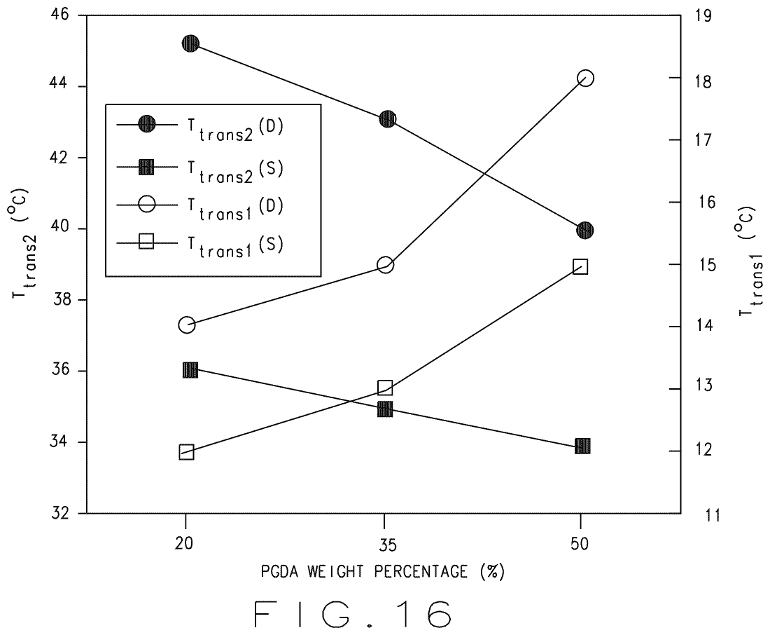

The swelling behavior of PGDA-PUA-PAA with varying PGDA:PUA:PAA weight ratios over 24 days is depicted in FIG. 15A. The sample with a weight ratio of 20:50:30 swells the most, reaching an equilibrium swelling ratio of >20 times, while the sample with a 35:35:30 ratio expands ˜10 times, and the 50:20:30 sample exhibits a swelling ratio of ˜5 times. These results suggest that a higher PGDA content which provides a higher crosslinking density can decrease the water absorption. PGDA-PUA-PAA demonstrates different swelling behaviors when exposed to PBS at 37° C., PBS at 25° C., and DI water at 25° C. (FIG. 15B). For the 50:20:30 sample the greatest swelling occurred in PBS at 37° C., making it particularly suitable for biomedical applications. The lower swelling ratios in DI water and at lower temperatures indicate that the ions and temperature of the solution may influence the swelling kinetics. The swelling of PGDA-PUA-PAA is not only transformed to a hydrogel but also plays a crucial role in the dynamic adjustment of the transition temperatures (Tₜᵣₐₙₛ₁ and Tₜᵣₐₙₛ₂). The results from DSC curves of samples with three PGDA:PUA:PAA weight ratios in both dry (D) and swelled (S) states are plotted in FIG. 16 and show that Tₜᵣₐₙₛ₂ of the dry samples are in 40° C.-45° C., which are slightly above the physiological temperature of 37° C. Upon swelling, Tₜᵣₐₙₛ₂ decrease to 34° C.-36° C. below 37° C. This triggers the second shape transition to its permanent shape in physiological conditions possible. A similar trend of reduced Tₜᵣₐₙₛ₁ upon hydration is observed. This hydrothermal activation is advantageous in controlling shape transitions within the body.

A key aspect of designing a SMP for biomedical applications is to optimize its mechanical properties to be compatible with biological tissues. Dynamic mechanical analysis (DMA) curves of PGDA-PUA-PAA in dry and swelled states show plateaus between Tₜᵣₐₙₛ₁ and Tₜᵣₐₙₛ₂, indicating that within this temperature range the storage modulus (E′) remains insensitive to temperature changes. This suggests that the material maintains a high elasticity between Tₜᵣₐₙₛ₁ and Tₜᵣₐₙₛ₂. Such behavior is beneficial for MIS during catheter operations, where a gradual temperature rise in the catheter is expected. This result is further confirmed by tensile testing. In a dry state, the materials containing 20 wt %, 35 wt %, and 50 wt % PGDA exhibit ultimate tensile strengths (σ_{T}) of 0.27 MPa, 1.04 MPa, and 2.16 MPa with fracture strains (ε_{f}) of 393%, 251%, and 163%, respectively. Strikingly, after hydration, all the PGDA-PUA-PAA hydrogel samples maintain elongations of >150% though their tensile stresses are reduced. This high elasticity helps to make the structure robust, which is vital for the long-term implantation success. Toughness (U_{T}) and Young's modulus (E), derived from the stress-strain curves were plotted. The U_{T} and E values in the dry state range from ˜0.4 MJ/m³ and ˜0.13 MPa for the sample with 20% PGDA to ˜1.6 MJ/m³ and ˜5.5 MPa for the sample with 50% PGDA, respectively. In a hydration state, Ur and E increase from ˜0.02 MJ/m³ and ˜0.02 MPa to ˜0.5 MJ/m³ and ˜2 MPa, respectively, for both types of samples. Compared to some biomaterials, the PGDA-PUA-PAA hydrogel demonstrates mechanical properties closer to the cardiovascular tissue. This improved mechanical compatibility with tissue is expected to better cell-material interactions, which will be discussed later.

Shape memory behavior of PGDA-PUA-PAA in a dry state was first characterized. The shape fixity ratio (R_{f}) and shape recovery ratio (Rᵣ) are assessed in a tensile mode at Tₜᵣₐₙₛ₁ and Tₜᵣₐₙₛ₂ for SMPs with varying PGDA ratios. The results show that R_{f} and Rᵣ of the temporary shape programmed at T of >Tₜᵣₐₙₛ₂ increases as the PGDA weight ratio increases, reaching ˜100% and ˜90% when PGDA is 50%, while the trend for the shape programmed at T (Tₜᵣₐₙₛ₂>T>Tₜᵣₐₙₛ₁) is opposite for Rᵣ which is reduced to ˜70% when PGDA is 50 wt % and R_{f} is quite stable at ˜75%. To illustrate independent activation at each transition temperature, a printed 3D model was programmed to sit at Tₜᵣₐₙₛ₂ and raise the arms at Tₜᵣₐₙₛ₁. This model highlights that activating the shape transition at one temperature does not inadvertently trigger the other, showcasing a distinct and sequential control over the shape memory behaviors. PGDA-PUA-PAA has potential in applications where complex shape transformations are required in response to natural triggers.

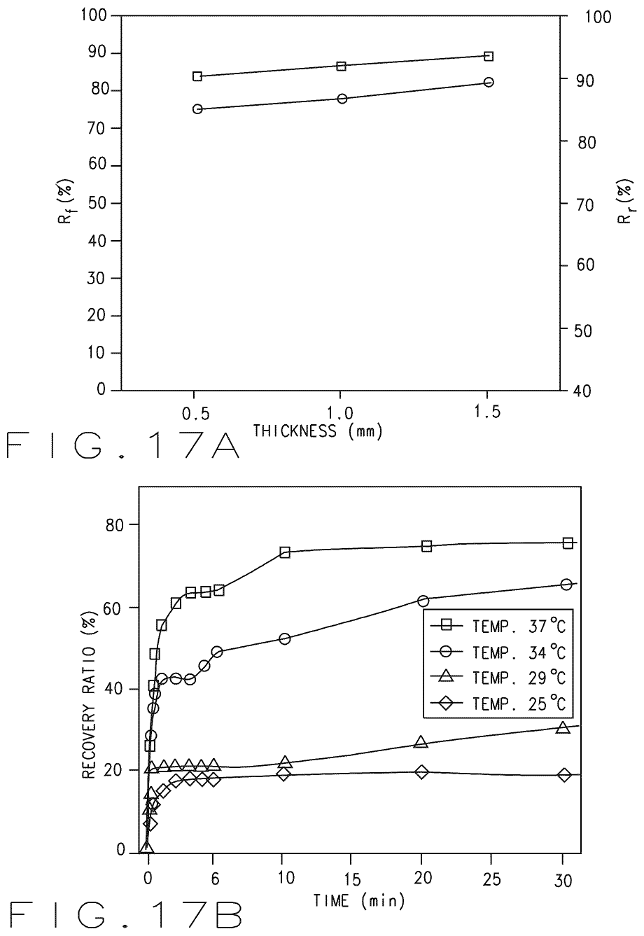

A unique behavior of this SMP is that it shows programmed recovery onset and transitioning to a SMP hydrogel upon swelling. FIG. 17A illustrates R_{f} and Rᵣ of SMP samples with varying thicknesses. The programmed shapes were fixed at room temperature (RT, Tₜᵣₐₙₛ₂>T>Tₜᵣₐₙₛ₁) and then stored at 4° C., and then recovered in PBS at 37° C. R_{f} and Rᵣ increase with thickness, ranging from ˜80% to 90%. Also studied were Rᵣ and recovery speed (Rₛ) in a bending mode for samples with different ratios and thicknesses programmed at RT (T>Tₜᵣₐₙₛ₁), fixed at T<Tₜᵣₐₙₛ₁ and recovered in 37° C. PBS. Rᵣ and Rₛ range from ˜90% to 100% and 0.25 rad/s to 9 rad/s, respectively. FIG. 17B illustrates the shape recovery profile of PGDA-PUA-PAA in PBS at 25° C., 29° C., 34° C., and 37° C. The recovery ratio increases as the temperature rises, with >75% recovery at ˜37° C. At lower temperatures of 25° C. and 29° C., the materials show 15-30% recovery over the same period. Interestingly, an intermediate onset at temperature between Tₜᵣₐₙₛ₁ and Tₜᵣₐₙₛ₂ is observed, where the shape recovery is not activated until the hydration leads to reduced Tₜᵣₐₙₛ₂ smaller than the set PBS temperature. The onset time varies with the temperature. At 25° C., the second recovery does not commence within 30 minutes whereas at 29° C., an onset period of ˜10 minutes is observed. At 34° C. and 37° C., the onset duration is ˜6 minutes.

To investigate the water-triggered shape memory behaviors and mechanical performance, the structural evolution was studied. The first hypothesis is that when the material is hydrated hydrogen bonds (H-bonds) between protons in —COOH and electronegative carbonyl groups in C═O or between AA-AA dimers are formed, which enhances the elasticity of the hydrogel. Water may also induce phase separation due to amphiphilic properties of the polymer network. In this process, PAA interacts with PGDA via the stable hydrogen bonding, which may form a hydrophilic PGDA-PAA phase separated from a hydrophobic PUA phase. This PUA phase may protect the formed H-bonds, allowing the material to maintain high elasticity, which contrasts with the fact that water, as a plasticizer, weakens most polymers upon water absorption. This observation is supported by earlier results. As the UA percentage increases from 20% to 50%, the swelling ratio increases by >3 times, but the elasticity of PGDA-PUA-PAA increases with the highest fracture strain in the 50% PUA sample. It is also hypothesized that this phase separation may lead to the formation of porous structures. To test the hypothesis of pore formation, first the microstructure evolution was investigated by Scanning Electron Microscopy (SEM) imaging of freeze-dried PGDA-PUA-PAA samples with varying UA weight ratios. Cross-sectional SEM images reveal porous structures with pore sizes of several micrometers. This indicates mild phase separation, largely due to H-bonds between PAA-PGDA and PAA-PAA. When the UA ratio is 20%, the SEM images display an aggregated structure, suggesting reduced porosity following PUA copolymerization. As the PUA ratio increases to 35%, honeycomb-like pores with 8.99±2.77 μm in size are formed, indicating notable phase separation. With 50% PUA ratio in the SMP, a snowflake-like porous structure emerges, comprising relatively uniform tens of micrometer sized pores. These self-assembled microstructures are controlled by the H-bonds between donor and acceptor components. These insights provide a useful strategy for developing robust hydrogels with micrometer porous structures.

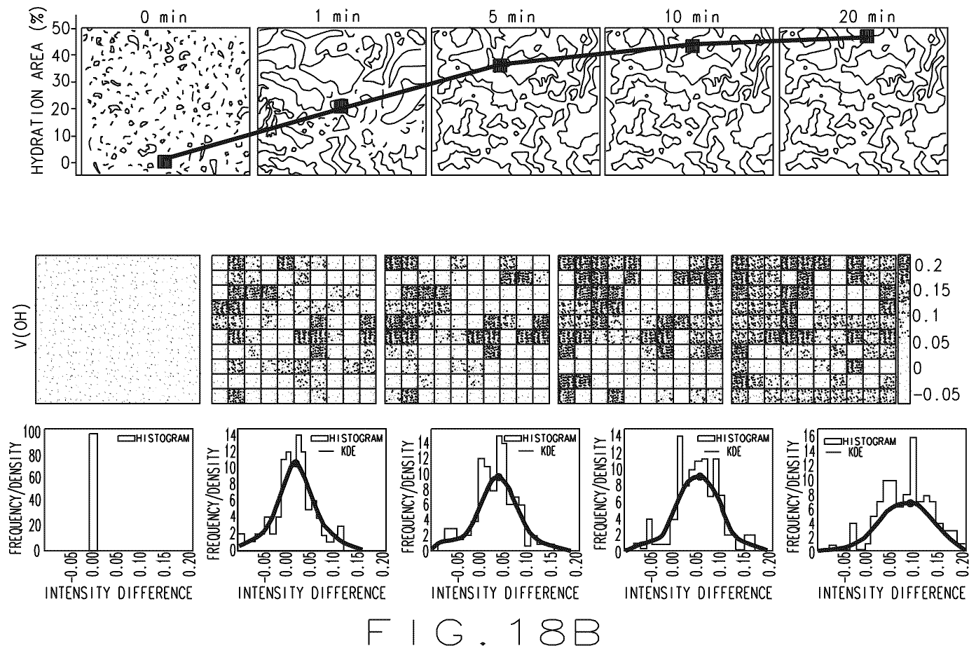

The structure evolution of the hydration-induced phase separation in the PGDA-PUA-PAA system was further characterized by spatiotemporal FTIR microscopy in a reflectance mode. FIG. 18A displays the evolving optical images of the samples where water diffuses. These images were first converted to grayscale followed by intensity transformation for enhanced visualization. Then these processed grayscale images are segmented to get the water distribution percentages. The percentages of white areas representing the evolved water regions are plot in FIG. 18A which shows an increase to ˜50% of the total area after 20-min water diffusion. Meanwhile, spatial FTIR spectra over these areas at each time step were also taken. To exclude the background information from the dry samples, a difference spectroscopy (DS) technique by subtracting a spectrum of a dry sample from that of the same sample after hydration in the ranges of interest (3,800-3,400 cm⁻¹ for v(OH)) was used to generate spatiotemporal v(OH) intensity difference maps as shown in FIG. 18B. In these images, a pixel with a deeper blue color represents a region with a higher concentration of H-bonds. After 1 minute of immersion in water, the sample begins to hydrate. These regions are depicted as patches in the v(OH) map, marking the initial stage of hydration. After 5 minutes, the v(OH) intensities increase, suggesting the formation of H-bonds. Combined with the optical image showing pore formation, it indicates a progressing phase separation of polymer segments in PGDA-PUA-PAA driven by H-bonding and amphiphilic interactions among the segments. In 10-20 minutes, the hydration-induced phase separation becomes stable with little increase in swelling. To correlate microscopic and chemical imaging, the FTIR images in FIG. 18B were also converted to grayscale followed by pixel intensity extraction along the selected lines. There is a strong alignment between optical and FTIR imaging data, underscoring the correlation between structural and chemical changes during hydration. To further quantify the H-bond evolution, the peak intensity distribution of the FTIR image at each time step is plot in the histogram images (FIG. 18B). From them one can analyze the Kernel Density Estimates (KDEs). Histograms provide frequency distributions, while KDEs show the probability density. The x-coordinates of the KDE peaks, representing the most likely intensity differences, were recorded to observe temporal changes across the v(OH) intensity difference shown in FIG. 18B. Quantitative analysis reveals that the peaks of the v(OH) intensity differences (x-axis values in FIG. 18B) shift towards higher values: 0.0, 0.0216, 0.0470, 0.0634, and 0.1034. This consistent increase suggests a gradual intensification of the OH bond distribution over time. This trend well agrees with the result shown in the average FTIR spectra over time, highlighting the dynamic evolution of water bonded to polymer chains. These results from the in-depth data analysis disclose the critical role of water in driving phase separation within PGDA-PUA-PAA, enabling its transition from a elastomer into a microporous shape memory hydrogel (SMH). The H-bond help the self-assembled phases maintain the material's elasticity, while the formed micropores in the hydrogel would facilitate nutrient transport, which benefits the tissue engineering.

### Example 22: PGDA-PUA-PAA Multi-Transition Implant

Benefit of the 4D printed PGDA-PUA-PAA was then demonstrated by showcasing its application for endovascular embolization. Before the implantation via MIS, biocompatibility of the material was first assessed by in vitro culturing with C3H10T1/2 cells. Fluorescence images of C3H10T1/2 cells cultured in dishes with PGDA-PUA-PAA with all three different ratios for 1, 2, 3, and 4 days were acquired, with living and dead cells stained blue and red, respectively. There was no significant difference in cell survival between the control and materials-co-cultured groups, indicating excellent biocompatibility of the material. Cell viability analysis revealed survival rates of >95% (death rates of <5%) after 4 days, suggesting great cytocompatibility. Characterizations have shown that this resin not only retains excellent printability, tunable phase transition temperatures, great mechanical properties and biocompatibility. The multi-transition behavior enables the programming of two distinct shapes, resulting in the shape recovery profile described above. Upon deployment in a physiological environment, the material transitions from a dry SMP to a SMH with well-maintained high elasticity. These advantageous properties are beneficial for an embolization application. The deployment process of a coil is described below. First, the printed coil is in a viscoelastic and deformable state at room temperature, and two different shapes are programmed. During the catheter transmission, the coil enters a transition state (Tₜᵣₐₙₛ₂>T>Tₜᵣₐₙₛ₁), where it elongates into a linear form—its first programmed shape. The well-maintained high elasticity facilitates the navigation through irregular vascular pathways. Once the coil reaches a target aneurysm site and is exposed to physiological conditions. Tₜᵣₐₙₛ₂ drops below body temperature, triggering the second shape transition, causing the linear shape to revert to its original coil shape followed by transformation into SMH, which ensures that the largely expanded coil conforms tightly to the aneurysm wall, providing complete occlusion and reducing the risk of migration or incomplete closure. The efficacy of the coil was experimentally tested in a simulated flow model designed for a blood vessel occlusion. The PGDA-PUA-PAA was utilized to fabricate the coils to fit the inner diameter of a 2 mm vascular replica tube, simulating a renal artery. The coils were programmed into a linear shape to fit the inner diameter of a clinical grade 4F catheter for delivery. The shape programming ensured their straight configuration for smooth catheter navigation and deployment in an in-vitro flow system that was designed to replicate physiological conditions. A peristaltic pump controlled with Arduino board was used to simulate blood flow, with flow rates set between 300 and 500 mL/min to match visceral artery flow dynamics. The system was immersed in phosphate-buffered saline (PBS) at 37° C. to mimic body temperature. The deployed coil was observed to expand and conform tightly to the vessel wall upon deployment, effectively occluding the flow, suggesting a success the fabricated device for potential vascular occlusion.

In view of the above, it will be seen that the several objects and advantages of the present invention have been achieved and other advantageous results have been obtained. The polymer and copolymers described herein, as well as their methods of production and control and the applications for them, constitute a significant advance in the ability to synthesize and use metal-free, high-capacity polymer materials.

As various changes could be made in the above constructions without departing from the scope of the invention, it is intended that all matter contained in the above description or shown in the accompanying drawings shall be interpreted as illustrative and not in a limiting sense.

What is claimed is:

1. A shape memory elastomer (SME), said SME comprising:
a copolymer network, the copolymer network comprising:
a first polymer, the first polymer being a biocompatible, biodegradable shape memory polymer with hydrophobic character; and
a second polymer bonded to the first polymer, the second polymer being a biocompatible, biodegradable hydrophilic polymer that has one or more functional groups capable of forming hydrogen bonds.

2. The SME of claim 1 wherein the first polymer is a polymer selected from the list consisting of poly(glycerol dodecanoate acrylate) (PGDA), polyurethane, polycaprolactone, polylactic acid, polyethylene, polystyrene, polynorborene, polymethyl methacrylate, polyvinyl chloride, poly(ethylene-co-vinyl acetate), and polyimide.

3. The SME of claim 1 wherein the one or more functional groups are selected from the list consisting of carboxylic acids, hydroxyls, amides, and phenols.

4. The SME of claim 3 wherein the second polymer is a polymer selected from the list consisting of polyacrylic acid (PAA), polyvinyl alcohol, polyethylene glycol, polyethylene glycol diacrylate, polyacrylamide, polymethacrylic acid, hyaluronic acid, chitosan, tannic acid, polyitaconic acid, maleic acid, alginate, peptide, chitosan, and collagen.

5. The SME of claim 1 wherein the first polymer is poly(glycerol dodecanoate acrylate) (PGDA) and the second polymer is polyacrylic acid (PAA), and further wherein the copolymer network is a PGDA-PAA copolymer network.

6. The SME of claim 5 wherein the PGDA-PAA copolymer network comprises PGDA and PAA in a PGDA:PAA weight ratio of 70:30.

7. The SME of claim 1 wherein the SME undergoes a shape change transition at a transition temperature Tₜᵣₐₙₛ between 30° C. and 50° C.

8. The SME of claim 7 wherein the SME undergoes a shape change transition at a transition temperature Tₜᵣₐₙₛ of approximately 39° C.

9. The SME of claim 1 further comprising a third polymer, wherein the third polymer is a biodegradable, biocompatible shape memory polymer that is different from the first polymer, and wherein the first polymer, the second polymer, and the third polymer are connected to one another.

10. The SME of claim 9 wherein the third polymer is a polymer selected from the list consisting of 10-undecenoic acid (UA), polyurethane, polycaprolactone, polylactic acid, polyethylene, polystyrene, polynorborene, polymethyl methacrylate, polyvinyl chloride, poly(ethylene-co-vinyl acetate), and polyimide.

11. The SME of claim 10 wherein the first polymer is poly(glycerol dodecanoate acrylate) (PGDA), the second polymer is polyacrylic acid (PAA), and the third polymer is 10-undecenoic acid (UA), wherein the copolymer network is a PGDA-PUA-PAA copolymer network.

12. The SME of claim 11 wherein the PGDA-PUA-PAA copolymer network comprises PGDA, PAA, and PUA in a PGDA:PUA:PAA weight ratio of 50:20:30.

13. An object comprising a shape memory elastomer (SME), said SME comprising:
a copolymer network, the copolymer network comprising:
a first polymer, the first polymer being a biocompatible, biodegradable shape memory polymer with hydrophobic character; and
a second polymer bonded to the first polymer, the second polymer being a biocompatible, biodegradable hydrophilic polymer that has one or more functional groups capable of forming hydrogen bonds;
further wherein the object has an original shape that is set during a synthesis of the object, and wherein the object:
can be deformed from the original shape into a first programmed shape above a first transition temperature and below a second transition temperature;
can be deformed into a second programmed shape below the first transition temperature, wherein the second transition temperature is higher than the first transition temperature, and
returns to the original shape above a second transition temperature.

14. The object of claim 13 wherein the first transition temperature is below 30° C. and wherein the second transition temperature is above 35° C.

15. The object of claim 13 wherein the object absorbs and retains water when in contact with water until the object becomes a hydrogel, and further wherein the first transition temperature and the second transition temperature decrease as the object absorbs more of the aqueous solution.

16. The object of claim 15 wherein when the object is dry, the second transition temperature is above an internal body temperature, and when the object is a hydrogel, the second transition temperature is below the internal body temperature.

17. The object of claim 15 wherein when the object is dry, the second transition temperature is above 40° C., and when the object is a hydrogel, the second transition temperature is below 40° C.

18. The object of claim 16 further comprising at least one type of particle selected from the list consisting of magnetically active particles and optically active particles.

19. A biomedical implant comprising a shape memory elastomer (SME), said SME comprising:
a copolymer network, the copolymer network comprising:
a first polymer, the first polymer being a biocompatible, biodegradable shape memory polymer with hydrophobic character; and
a second polymer bonded to the first polymer, the second polymer comprising a biocompatible, biodegradable hydrophilic polymer that has one or more functional groups capable of forming hydrogen bonds;
wherein the biomedical implant has an original shape, wherein the original shape is set during a synthesis of the biomedical implant, and wherein the original shape can support bodily tissue at a treatment site in a patient.

20. The biomedical implant of claim 19 wherein the biomedical implant can be delivered to a treatment site via a catheter.

21. A method of manufacturing a shape memory elastomer (SME), said SME comprising:
a copolymer network, the copolymer network comprising:
a first polymer, the first polymer being a biocompatible, biodegradable shape memory polymer with hydrophobic character; and
a second polymer bonded to the first polymer, the second polymer being a biocompatible, biodegradable hydrophilic polymer that has one or more functional groups capable of forming hydrogen bonds;
wherein the method of printing is selected from the list consisting of: molding, extrusion, machining, extrusion-based 3D printing, and light-based 3D printing.
